(19) United States
(12) Patent Application Publication
(10) Pub. No.: US 2006/0206209 A1
(43) Pub. Date: Sep. 14, 2006

(54) Prosthetic nucleus apparatus and methods

(76) Inventors: Andrew H. Cragg, Edina, MN (US); Robert L. Assell, Wilmington, NC (US); Bradley J. Wessman, Wilmington, NC (US)

Correspondence Address: KONDZELLA AND CYR, PONDVIEW PLAZA, SUITE 114, 5850 OPUS PARKWAY, MINNETONKA, MN, 55343, US

(21) Appl. No.: 11/199,541

(22) Filed: Aug. 8, 2005

Related U.S. Application Data
(63) Continuation-in-part of application No. 10/972,184, filed on Oct. 22, 2004.
(63) Continuation-in-part of application No. 10/972,039, filed on Oct. 22, 2004.
(63) Continuation-in-part of application No. 10/972,040, filed on Oct. 22, 2004.
(63) Continuation-in-part of application No. 10/972,176, filed on Oct. 22, 2004.
(63) Continuation-in-part of application No. 10/972,065, filed on Oct. 22, 2004.
(63) Continuation-in-part of application No. 10/971,781, filed on Oct. 22, 2004.
(63) Continuation-in-part of application No. 10/971,731, filed on Oct. 22, 2004.
(63) Continuation-in-part of application No. 10/972,077, filed on Oct. 22, 2004.
(63) Continuation-in-part of application No. 10/971,765, filed on Oct. 22, 2004.
(63) Continuation-in-part of application No. 10/971,775, filed on Oct. 22, 2004.
(63) Continuation-in-part of application No. 10/972,299, filed on Oct. 22, 2004.
(63) Continuation-in-part of application No. 10/971,780, filed on Oct. 22, 2004.
(60) Provisional application No. 60/558,069, filed on Mar. 31, 2004.
(60) Provisional application No. 60/513,899, filed on Oct. 23, 2003.
(60) Provisional application No. 60/599,989, filed on Aug. 9, 2004.

(51) Int. Cl. A61F 2/44

(52) U.S. Cl. 623/17.16

(57) ABSTRACT

Prosthetic nucleus apparatus and methods for treating an intervertebral disc are disclosed. Prosthetic nucleus apparatus may include a barrier sealant membrane and a prosthetic nucleus material. The barrier sealant membrane forms a chamber which can receive the prosthetic nucleus material. The barrier sealant membrane can be formed by depositing a layer of material on a tissue surface within a de-nucleated space within an intervertebral disc. The prosthetic nucleus material may be positioned within the chamber of the barrier sealant membrane after the barrier sealant membrane is deposited within the de-nucleated space. The barrier sealant membrane and the prosthetic nucleus material may be positioned within a patient through an axial trans-sacral bore. A plug may also be included to prevent expulsion of the barrier sealant membrane and prosthetic nucleus material.

### CROSS-REFERENCE TO RELATED APPLICATIONS

The present U.S. patent application claims priority and benefits from co-pending and commonly assigned U.S. Prov. Pat. Appl. No. 60/599,989 filed Aug. 9, 2004, and is a continuation-in-part of co-pending U.S. patent application Ser. Nos. 10/972,184; 10/972,039; and 10/972,040; 10/972,176; and U.S. application Ser. Nos. 10/972,065; 10/971,779; 10/971,781; 10/971,731; 10/972,077; 10/971,765;10/971,775; 10/972,299; 10/971,780; all of which were filed on Oct. 22, 2004 and which claim priority and benefits from U.S. Provisional Patent Application Nos. 60/558,069 filed Mar. 31, 2004 and 60/513,899 filed Oct. 23, 2003, which claim the benefit of priority from commonly assigned U.S. Pat. No. 6,921,403 “Method and Apparatus for Spinal Distraction and Fusion” issued on Jul. 26, 2005, which is a continuation-in-part of commonly assigned U.S. Pat. No. 6,899,716 “Method and Apparatus for Spinal Augmentation” issued on May 31, 2005, which is a continuation-in-part of U.S. patent application Ser. No. 09/848,556, filed on May 3, 2001, which is a continuation-in-part of commonly assigned U.S. Pat. No. 6,558,390 “Methods and Apparatus for Performing Therapeutic Procedures in the Spine,” and co-pending U.S. patent application Ser. No. 10/459,149, filed on Jun. 10, 2003, which is a continuation of commonly assigned U.S. Pat. No. 6,575,979 “Method and Apparatus for Providing Posterior or Anterior Trans-Sacral Access to Spinal Vertebrae,” issued on Jun. 10, 2003; and is commonly owned along with U.S. Pat. No. 6,558,386 “Axial Spinal Implant and Method and Apparatus for Implanting an Axial Spinal Implant within the Vertebrae of the Spine,” issued May 6, 2003 each of which claim priority to U.S. Provisional patent Application No. 60/182,748, filed on Feb. 16, 2000. The contents of each of the aforementioned U.S. patents and patent applications are hereby incorporated in their entirety into this disclosure by reference.

### BACKGROUND OF THE INVENTION

1. Field of the Invention

The present invention relates to prosthetic nucleus apparatus and, more particularly, to prosthetic nucleus apparatus that may be introduced percutaneously using minimally invasive techniques to provide therapy to the spine.

2. Description of the Related Art

There are currently over 700,000 surgical procedures performed annually to treat lower back pain in the U.S. In 2004, it is conservatively estimated that there will be more than 200,000 lumbar fusions performed in the U.S., and more than 300,000 worldwide, representing approximately a $1,000,000,000.00 endeavor in an attempt to alleviate patients' pain. Approximately 60% of spinal surgery takes place in the lumbar spine, and of that portion approximately 80% involves the lower lumbar vertebrae designated as the fourth lumbar vertebra (“L4”), the fifth lumbar vertebra (“L5”), and the first sacral vertebra (“S1”). Persistent low back pain is often attributable to degeneration of the intervertebral disc between L5 and S1.

Traditional, conservative methods of treatment include bed rest, pain and muscle relaxant medication, physical therapy or steroid injection. Upon failure of conservative therapy spinal pain has traditionally been treated by surgical interventions, e.g., spinal arthroplasty; arthrodesis, or fusion, which causes the vertebrae above and below the intervertebral disc to grow solidly together and form a single, solid piece of bone. Yet, statistics show that only about 70% of these procedures performed will be successful in relieving pain. Thus, the market for intervertebral disc replacement and repair is expected to grow even more rapidly than other treatments as new techniques and Devices are approved.

Within the overall spine arena, it is estimated that the potential market for treatment or replacement of intervertebral discs will surpass $1 billion by 2007. Moreover, there may be multiple causes (e.g., exertion or aging) of patients' lower back pain, where the pain generators are hypothesized to include one or more of the following: bulging of the posterior annulus fibrosus or PLL with subsequent nerve impingement; tears, fissures or cracks in the outer, innervated layers of the annulus fibrosus; motion induced leakage of nuclear material through the annulus fibrosus and subsequent irritation of surrounding tissue in response to the foreign body reaction, or facet pain. Generally it is believed that 75% of cases are associated with degenerative disc disease, where the intervertebral disc of the spine suffers reduced mechanical functionality. Surgical procedures, such as spinal fusion and discectomy, may alleviate pain, but do not restore the normal physiological intervertebral disc function attributable to healthy anatomical form, i.e., intact intervertebral disc structures such as the nucleus pulposus and annulus fibrosus fibrosis, as described below.

The spinal column or backbone encloses the spinal cord and consists of 33 vertebrae superimposed upon one another in a series which provides a flexible supporting column for the trunk and head. The vertebrae cephalad (i.e., toward the head or superior) to the sacral vertebrae are separated by fibrocartilaginous intervertebral discs and are united by articular capsules and by ligaments. The uppermost seven vertebrae are referred to as the cervical vertebrae, and the next lower twelve vertebrae are referred to as the thoracic, or dorsal, vertebrae. The next lower succeeding five vertebrae below the thoracic vertebrae are referred to as the lumbar vertebrae and are designated L1-L5 in descending order. The next lower succeeding five vertebrae below the lumbar vertebrae are referred to as the sacral vertebrae and are numbered S1-S5 in descending order. The final four vertebrae below the sacral vertebrae are referred to as the coccygeal vertebrae. In adults, the five sacral vertebrae fuse to form a single bone referred to as the sacrum, and the four rudimentary coccyx vertebrae fuse to form another bone called the coccyx or commonly the “tail bone”. The number of vertebrae is sometimes increased by an additional vertebra in one region, and sometimes one may be absent in another region.

The bodies of successive lumbar, thoracic and cervical vertebrae articulate with one another and are separated by the intervertebral discs. Each intervertebral disc includes a fibrous cartilage shell enclosing a central mass, the “nucleus pulposus” (or “nucleus pulposus” herein) that provides for cushioning and dampening of compressive forces to the spinal column. The shell enclosing the nucleus pulposus includes cartilaginous endplates adhered to the opposed cortical bone endplates of the cephalad and caudal vertebral bodies and the “annulus fibrosus fibrosis” (or “annulus fibrosus” herein) including multiple layers of opposing collagen fibers running circumferentially around the nucleus pulposus and connecting the cartilaginous endplates. The natural, physiological nucleus pulposus is included of hydrophilic (water attracting) mucopolysacharides and fibrous strands (protein polymers). The nucleus pulposus is relatively inelastic, but the annulus fibrosus can bulge outward slightly to accommodate loads axially applied to the spinal motion segment. The intervertebral discs are anterior to the spinal canal and located between the opposed end faces or endplates of a cephalad and a caudal vertebral bodies. The inferior articular processes articulate with the superior articular processes of the next succeeding vertebra in the caudal (i.e., toward the feet or inferior) direction. Several ligaments (supraspinous, interspinous, anterior and posterior longitudinal, and the ligamenta flava) hold the vertebrae in position yet permit a limited degree of movement. The assembly of two vertebral bodies, the interposed, intervertebral, disc and the attached ligaments, muscles and facet joints is referred to as a “spinal motion segment”.

The relatively large vertebral bodies located in the anterior portion of the spine and the intervertebral discs provide the majority of the weight bearing support of the vertebral column. Each vertebral body has relatively strong, cortical bone layer including the exposed outside surface of the body, including the endplates, and weaker, cancellous bone including the center of the vertebral body.

The nucleus pulposus that forms the center portion of the intervertebral disc consists of 80% water that is absorbed by the proteoglycans in a healthy adult spine. With aging, the nucleus pulposus becomes less fluid and more viscous and sometimes even dehydrates and contracts (sometimes referred to as “isolated disc resorption”) causing severe pain in many instances. The intervertebral discs serve as “dampeners” between each vertebral body that minimize the impact of movement on the spinal column, and disc degeneration, marked by a decrease in water content within the nucleus pulposus, renders intervertebral discs ineffective in transferring loads to the annulus fibrosus layers. In addition, the annulus fibrosus tends to thicken, desiccate, and become more rigid, lessening its ability to elastically deform under load and making it susceptible to fracturing or fissuring, and one form of degeneration of the intervertebral disc thus occurs when the annulus fibrosus fissures or is torn. A fissure may or may not be accompanied by extrusion of nucleus pulposus material into and beyond the annulus fibrosus. The fissure itself may be the sole morphological change, above and beyond generalized degenerative changes in the connective tissue of the intervertebral disc, and intervertebral disc fissures can nevertheless be painful and debilitating. Biochemicals contained within the nucleus pulposus may escape through the fissure and irritate nearby structures.

A fissure also may be associated with a herniation or rupture of the annulus fibrosus causing the nucleus pulposus to bulge outward or extrude out through the fissure and impinge upon the spinal column or nerves (a “ruptured” or “slipped” disc). With a contained intervertebral disc herniation, the nucleus pulposus may work its way partly through the annulus fibrosus but is still contained within the annulus fibrosus or beneath the posterior longitudinal ligament, and there are no free nucleus pulposus fragments in the spinal canal. Nevertheless, even a contained intervertebral disc herniation can be problematic because the outward protrusion can press on the spinal cord or on spinal nerves causing sciatica.

Another intervertebral disc problem may occur when the intervertebral disc bulges outward circumferentially in all directions and not just in one location. This occurs when, over time, the intervertebral disc weakens bulges outward and takes on a “roll” shape. Mechanical stiffness of the joint is reduced and the spinal motion segment may become unstable, shortening the spinal cord segment. As the intervertebral disc “roll” extends beyond the normal circumference, the intervertebral disc height may be compromised, and foramina with nerve roots are compressed causing pain. Current treatment methods other than spinal fusion for symptomatic intervertebral disc rolls and herniated intervertebral discs include “laminectomy” which involves the surgical exposure of the annulus fibrosus and surgical excision of the symptomatic portion of the herniated intervertebral disc followed by a relatively lengthy recuperation period. In addition, osteophytes may form on the outer surface of the intervertebral disc roll and further encroach on the spinal canal and foramina through which nerves pass. The cephalad vertebra may eventually settle on top of the caudal vertebra. This condition is called “lumbar spondylosis”. Various other surgical treatments that attempt to preserve the intervertebral disc and to simply relieve pain include a “discectomy” or “disc decompression” to remove some or most of the interior nucleus pulposus thereby decompressing and decreasing outward pressure on the annulus fibrosus. In less invasive microsurgical procedures known as “microlumbar discectomy” and “automated percutaneous lumbar discectomy”, the nucleus pulposus is removed by suction through a needle laterally extended through the annulus fibrosus. Although these procedures are less invasive than open surgery, they nevertheless suffer the possibility of injury to the nerve root and dural sac, perineural scar formation, re-herniation of the site of the surgery, and instability due to excess bone removal. In addition, they generally involve the perforation of the annulus fibrosus.

Although damaged intervertebral discs and vertebral bodies can be identified with sophisticated diagnostic imaging, existing surgical interventions so extensive and clinical outcomes are not consistently satisfactory. Furthermore, patients undergoing such fusion surgery experience significant complications and uncomfortable, prolonged convalescence. Surgical complications include intervertebral disc space infection; nerve root injury; hematoma formation; instability of adjacent vertebrae, and disruption of muscle, tendons, and ligaments, for example. Several companies are pursuing the development of prosthesis for the human spine, intended to completely replace a physiologic disc, i.e., an artificial disc. In individuals where the degree of degeneration has not progressed to destruction of the annulus fibrosus, rather than a total artificial intervertebral disc replacement, a preferred treatment option may be to replace or augment the nucleus pulposus, involving the deployment of a prosthetic nucleus pulposus.

As noted previously, the normal nucleus pulposus is contained within the space bounded by the bony vertebrae above and below it and the annulus fibrosus, which circumferentially surrounds it. In this way the nucleus pulposus is completely encapsulated and sealed with the only communication to the body being a fluid exchange that takes place through the bone interface with the vertebrae, known as the endplates. The hydroscopic material including the physiological nucleus pulposus has an affinity for water which is sufficiently powerful to distract (i.e., elevate or “inflate”) the intervertebral disc space, despite the significant physiological loads that are carried across the intervertebral disc in normal activities. These forces, which may range from about 0.4× to about 1.8× body weight, generate local pressure well above normal blood pressure, and the nucleus pulposus and inner annulus fibrosus tissue are, in fact, effectively avascular. The existence of the nucleus pulposus as a cushion (e.g., the nucleus pulposus is the “air” in the “tire” known as an intervertebral disc), and the annulus fibrosus, as a flexible member, contributes to the range of motion in the normal intervertebral disc. Range of motion is described in terms of degrees of freedom (i.e., translation and rotation about three orthogonal planes relative to a reference point, the instantaneous center of rotation around the vertical axis of the spine).

Compression of the spine is due to body weight and loads applied to the spine. Body weight is a minor compressive load. The major compressive load on the spine is produced by the back muscles. As a person bends forward, the body weight plus an external load must be balanced by the force generated by the back muscles. That is, muscle loads balance gravitational loads so that the spine is in equilibrium, to preclude us from falling over. The external force is calculated by multiplying the load times the perpendicular distance of the load from the spine. The greater the distance is from the spine, the larger the load is on the spine. Since the back muscles act close to the spine, they must exert large forces to balance the load. The force generated by the back muscles results in compression of spinal structures. Most of the compressive loads (˜80%) are sustained by the anterior column (intervertebral disc and vertebral body).

The intervertebral disc is, at least in part, a hydrostatic system. The nucleus pulposus acts as a confined fluid within the annulus fibrosus. The nucleus pulposus converts compressive on the vertebral end plates (axial loads) into tension on the fibers of the annulus fibrosus. Compression injuries occur by two main mechanisms; axial loading by gravity or by muscle action. Gravitational injuries result from a fall onto the buttocks while muscular injuries result from severe exertion during pulling or lifting. A serious consequence of the injury is a fracture of the vertebral end plate. Since the end plate is critical to disc nutrition, an injury can change the biochemical and metabolic state of the intervertebral disc. If the end plate heals, the intervertebral disc may suffer no malice. However, if the end plate does not heal, the nucleus pulposus can undergo harmful changes. The nucleus pulposus loses its proteoglycans and thus its water-binding capacity. The hydrostatic properties of the nucleus pulposus are compromised. Instead of sharing the load between the nucleus pulposus and the annulus fibrosus, more load is transferred to the annulus fibrosus. The fibers of the annulus fibrosus may then fail. In addition to annular tears, the layers of the annular separate (delaminate). The intervertebral disc may collapse or it may maintain its height with progressive annular tearing. If the annulus fibrosus is significantly weakened, there may be a rupture of the intervertebral disc whereby the nuclear material migrates into the annulus fibrosus or into the spinal canal causing nerve root compression.

In the context of the present disclosure, the term distraction refers procedurally to an elevation in height that increases the intervertebral disc space which may result from introduction of the prosthetic nucleus apparatus 10s. This distraction may be achieved either in the axial deployment of a prosthetic nucleus apparatus 10 itself, or assisted by means of a temporary distraction rod, during implantation. Temporary distraction refers to elevation of intervertebral disc height by means, such as a distraction rod, which is subsequently removed but wherein the elevation is retained intra-operatively, while the patient remains prone. Thus, a device may be inserted into an elevated intervertebral disc space first created by other distraction means, and thereafter, the physical presence and dimensionality of the inserted device may preserve that height space. Doing so, the device may decompress the intervertebral disc and alleviate pain caused by nerve impingement

To date, drawbacks of related, contemplated or deployed, devices include subsidence; their tendency to extrude or migrate; to erode the bone; to degrade with time, or to fail to provide sufficient biomechanical load distribution and support. As noted previously, some of the drawbacks relate to the fact that the related devices deployment typically involves a virtually complete discectomy achieved by instruments introduced laterally through the patient's body to the intervertebral disc site and manipulated to cut away or drill lateral holes through the intervertebral disc and adjoining cortical bone. The endplates of the vertebral bodies, which include very hard cortical bone and help to give the vertebral bodies needed strength, are usually weakened or destroyed during the drilling. The vertebral endplates are special cartilage structures that surround the top and bottom of each vertebra and are in direct contact with the intervertebral disc. They are important to the nutrition of the intervertebral disc because they allow the passage of nutrients and water into the intervertebral disc. If these structures are injured, it can lead to deterioration of the intervertebral disc and altered intervertebral disc function. Not only do the large laterally drilled hole or holes compromise the integrity of the vertebral bodies, but the spinal cord can be injured if they are drilled too posteriorly.

Alternatively, related devices are sometimes deployed through a surgically created or enlarged hole in the annulus fibrosus. The annulus fibrosus consists of tough, thick collagen fibers. The collagen fibers which include the annulus fibrosus are arranged in concentric, alternating layers. Intra-layer orientation of these fibers is parallel, however, each alternating (i.e., interlayer) layers' collagen fibers are oriented obliquely (˜120′). This oblique orientation allows the annulus fibrosus to resist forces in both vertical and horizontal directions. Axial compression of an intervertebral disc results in increased pressure in the intervertebral disc space. This pressure is transferred to the annulus fibrosus in the form of loads (stresses) perpendicular to the wall of the annulus fibrosus. With applied stress, these fibrous layers are put in tension and the angle from horizontal decreases to better resist the load, i.e., the annulus fibrosus works to resist these perpendicular stresses by transferring the loads around the circumference of the annulus fibrosus (Hoop Stress). Vertical tension resists bending and distraction (flexion and extension). Horizontal tension resists rotation and sliding (i.e., twisting). While the vertical components of the annulus fibrosus' layers enable the intervertebral disc to withstand forward and backward bending well, only half of the horizontal fibers of the annulus fibrosus are engaged during a rotational movement. In general, the intervertebral disc is more susceptible to injury during a twisting motion, deriving its primary protection during rotation from the posterior facet joints; however, this risk is even greater if and when the annulus fibrosus is compromised. Moreover, annulus fibrosus disruption will remain post-operatively, and present a pathway for Devices extrusion and migration in addition to compromising the physiological biomechanics of the intervertebral disc structure.

Other devices, in an attempt to provide sufficient mechanical integrity to withstand the stresses to which they will be subjected, are configured to be so firm, stiff, and inflexible that they tend to erode the bone or become imbedded, over time, in the vertebral bodies, a phenomenon known as “subsidence”, sometimes also termed “telescoping”. The result of subsidence is that the effective length of the vertebral column is shortened, which can subsequently cause damage to the nerve root and nerves that pass between the two adjacent vertebrae.

In the context of the present disclosure, “biomechanics” refers to physiological forces on intervertebral disc structures (individually and collectively) attributable to movement of the lumbar spine, described in the previous explanation of the six degrees of freedom which include spinal range of motion. Further, in the context of the present disclosure, “dynamic” refers to devices with an inherent ability to allow mobility by enabling or facilitating forces or load bearing that assist or substitute for physiological structures that are otherwise compromised, weakened or absent.

### SUMMARY OF THE INVENTION

It may be an advantage of the present invention that the risks as described in the preceding Background of the Invention are less for the binary prosthetic nucleus apparatus due to: a) its atraumatic, annulus fibrosus-sparing, trans-sacral axial delivery; and b) the incorporation of barrier-sealant-matrix means. The barrier sealant membrane may repair tissue and/or seal existing fissures in the annulus fibrosus thereby retaining bulk prosthetic nucleus material within the intervertebral disc space. Additional retention of the prosthetic nucleus material within the intervertebral disc space may be further assured by a plug to seal at least one axial access tract into at least one vertebral body through which the components of the binary prosthetic nucleus apparatus were deployed.

The present invention provides a prosthetic nucleus apparatus. The prosthetic nucleus apparatus in accordance with the present invention may generally include materials and/or components that are positioned in the de-nucleated intervertebral disc space to augment or replace the nucleus pulposus of a de-nucleated intervertebral disc. Prosthetic nucleus apparatus can be introduced in situ within the spine, following a nucleectomy procedure. The introduction of the prosthetic nucleus apparatus may utilize a cannula that is introduced via a trans-sacral axial bore through the vertebral bodies into a surgically de-nucleated intervertebral disc space. Depending on the condition of the patient, prosthetic nucleus apparatus may be introduced into one or more intervertebral discs along the spine. In one aspect, a plurality of prosthetic nucleus apparatus may be introduced into adjacent motion segments' intervertebral discs. In doing so, the prosthetic nucleus apparatus may facilitate pain relief and preserve and/or restore the function of the intervertebral disc.

In one aspect of the present invention, an insoluble, non-degradable prosthetic nucleus apparatus is configured as a binary implant, i.e., including two structural components. More specifically, bulk prosthetic nucleus material(s) that can be dispensed via minimally invasive, atraumatic means within a de-nucleated intervertebral disc space into which a compliant barrier-sealant-membrane component may first be deployed in one or more layers to conformably contact and seal the interior disc surfaces (e.g., the annulus fibrosus and the disc endplates) and which barrier sealant membrane serves to preclude leakage, migration or expulsion through these structures (e.g., through fissures, as herniations) to contain the bulk prosthetic nucleus material within the intervertebral disc space, thereby assuring the ongoing ability of the prosthetic nucleus apparatus to functionally reproduce the same load-bearing characteristics as the natural intervertebral disc's nucleus pulposus, to preserve and/or restore mobility. More specifically, in an exemplary aspect of the invention, the barrier sealant membrane can be formed in vivo by using an in situ cure to serve as a tissue-cohesive interface between the anatomical structures, e.g., the annulus fibrosus and the disc endplates, and the bulk PNM dispensed into the interior disc space. In a preferred aspect, the barrier sealant membrane serves to seal, treat (e.g., via release of biosoluble therapeutic agents included among its component materials) and/or repair (e.g., by means of matrix incorporation of biopolymers, or proteins, included among its component materials) tissue, e.g., fissures in the annulus fibrosus; and serves as a semi-permeable membrane, i.e., a barrier to the migration or leakage of bulk prosthetic nucleus material and deleterious residual cross-linkers through the interface, while permitting the ingress and egress of physiologic fluids to maintain intervertebral disc hydration and the ability to transfer loads by means of hydrostatic forces.

In an aspect of the present invention, the prosthetic nucleus apparatus may be configured as a binary apparatus including a barrier sealant membrane and a prosthetic nucleus material. The barrier sealant membrane formed in situ within or with the tissue surfaces of the de-nucleated intervertebral disc space. The barrier sealant membrane may be formed in a configuration and/or from a material that is permeable or impermeable. The barrier sealant membrane defines a chamber that contains prosthetic nucleus pulposus material. The prosthetic nucleus material is typically dispensed into the chamber by injection or infusion. The barrier sealant membrane and prosthetic nucleus material components of the prosthetic nucleus apparatus may alone or in combination assist in distraction (i.e., restoring intervertebral disc height). In addition or alternatively, the barrier sealant membrane and prosthetic nucleus material components of the prosthetic nucleus apparatus may alone or in combination be configured to have desired viscoelastic properties. These viscoelastic properties can include bulk and compressive moduli for example. In one aspect, the bulk and compressive moduli may be designed to substantially “match” those characteristics of a native healthy nucleus pulposus. In other aspects, the barrier sealant membrane may be configured to functionally enable conformal contact of maximum surface area within the intervertebral disc space of a de-nucleated intervertebral disc. In still other aspects, the prosthetic nucleus apparatus may be configured to “mimic” physiologic load distribution and dissipation, prevent bone erosion or implant subsidence, and/or to exhibit sufficient resistance to fatigue and shear forces to preclude material fragmentation and migration out of the intervertebral disc.

Similarly, to contain the bulk prosthetic nucleus material within the intervertebral disc space, thereby assuring the ongoing ability of the prosthetic nucleus apparatus to functionally, substantially mimic the same load-bearing characteristics as the natural intervertebral disc's nucleus pulposus, following trans-sacral, axial access and deployment of the inventive binary prosthetic nucleus apparatus into the intervertebral disc space, to augment or replace the nucleus pulposus, the access tract can be mechanically sealed. Any one of numerous valve configurations, e.g., self-sealing valve assemblies or flow-stop devices may suitably serve this function. For example, a rod or threaded plug, inserted into the proximal end of the inferior vertebral body of the motion segment of the intervertebral disc into which the prosthetic nucleus apparatus is deployed, which plug extends sufficiently through and into the vertebral body may now serve as a stop flow Apparatus to preclude leakage, migration, or expulsion of prosthetic nucleus pulposus materials from the axial access bore to the intervertebral disc space. Materials suitable as plugs, such as non-absorbable threaded plugs, including those fabricated from medical grade polyether-ether-ketone (PEEK) such as that commercially available from Invibio Inc., in Lancashire, United Kingdom, or polyether-ketone-ketone (PEKK) available from Coors-Tech Corporation, in Colorado, or alternatively, conventional polymethylmethacrylate (PMMA); ultra high molecular weight polyethylene (UHMWPE), or other suitable polymers in combination with autologous or allograft bone dowels may be used as plugs.

The introduction of the binary prosthetic nucleus apparatus of the present invention may be accomplished without the need to surgically create or deleteriously enlarge an existing hole in the annulus fibrosus of the intervertebral disc. Such a creation or enlargement of an existing hole increases the risks of expulsion, migration, or subsidence of a prosthetic nucleus apparatus. As will be noted by those skilled in the art, prosthetic nucleus apparatus in accordance with the present invention are inherently less susceptible to expulsion, migration, or subsidence. Further, the deploying of the disclosed prosthetic nucleus apparatus may preserve or restore patients' mobility by relieving pain and/or more properly distributing physiological loads along the spine. This may be accomplished by distraction and decompression of the intervertebral disc during and/or after implantation of a prosthetic nucleus apparatus in accordance with the present invention.

Mobility preservation apparatus 10 provide dynamic stabilization across a progression-of-treatment interventions for treating symptomatic discogenic pain, ranging from treatment in patients where little degeneration or collapse is evident radio-graphically, to those for whom prosthetic nucleus apparatus 10 or total disc replacements are indicated. Total disc replacement would be indicated with more advanced disease than with a prosthetic nucleus apparatus 10, but where some annular function remains.

Prosthetic nucleus apparatus 10 may be indicated in patients with a greater degree of degeneration and loss of intervertebral disc height but not to the stage where advanced annular break-down is present, clinically indicating total disc replacement. Prosthetic nucleus apparatus 10 typically go beyond dynamic stabilization by generally including a complete nucleectomy and subsequent filling of the de-nucleated space with an appropriate material. Generally, the goal is to restore, as opposed to preserve, intervertebral disc height and motion.

One object of the present invention can be to provide alternative options for treating intervertebral disc degeneration when arthrodesis, i.e., fusion, is deemed too radical an intervention based on an assessment of the patient's age, degree of intervertebral disc degeneration, and prognosis. Specifically, the present invention may include an axially deployed spinal prosthetic nucleus apparatus which can provide discogenic pain relief by elevating and maintaining intervertebral disc height (distraction); by preserving or restoring mobility, and by substantially improving biomechanical function as compared to other methods and devices.

In a preferred aspect of the present invention, binary prosthetic nucleus apparatus are deployed into the intervertebral disc space in a minimally traumatic fashion via a trans-sacral, axial approach rather than laterally through the annulus fibrosus, without compromising it anatomically or functionally impairing its physiological load sharing, e.g., hoop stress response, as previously described. The binary prosthetic nucleus apparatus can include a barrier sealant membrane that is advantageous in repairing or sealing fissures or herniations in the annulus fibrosus, i.e., when it is not fully intact, which reduces risks of Apparatus expulsion or migration, e.g., laterally through an existing hole or fissure in the annulus fibrosus.

The prosthetic nucleus material may be dispensed into an intervertebral disc space following in situ cure and formation in vivo of the barrier sealant membrane. Upon deployment and formation of the prosthetic nucleus apparatus, it will be understood that that the barrier sealant membrane and prosthetic nucleus material may or may not remain as discernibly distinct. Particularly, the barrier sealant membrane and prosthetic nucleus material may be substantially the same component materials and, after formation, may not include a readily distinguishable interface. It is further will be understood that either or both barrier sealant membrane and prosthetic nucleus material components may themselves be configured as sub-assemblies, e.g., including a plurality of component materials, and that the formation or reconstitution of the components may involve intermediate processes or agents (e.g., surfactants; cross-linking agents; viscosity agents; buffers, etc.). It will also be understood that the barrier sealant membrane, in whole or in part, may be designed to be bioabsorbable or non-degradable as clinically indicated, while the prosthetic nucleus material is generally insoluble and non-degradable (and hence, so too, is the binary prosthetic nucleus apparatus).

In addition to biocompatibility, in another aspect of the present inventions, the materials of the prosthetic nucleus apparatus and its formation may be sterilizable, visible and/or imageable, e.g., fluoroscopically; or via computed tomography (CT), or magnetic resonance imaging (MRI), with this last-named imaging technique mandating that materials be substantially free of iron (Fe). Moreover, in consideration of contrast, detail, and spatial sensitivity, it is preferred that contrast media (e.g., iodine) or other materials (e.g., compounds including Tantalum; Titanium; or barium sulfate) be employed in configuring prosthetic nucleus apparatus when and where needed and appropriate, to supplement or modify radiolucency or radio-opaqueness.

In one aspect of the present invention, prosthetic nucleus apparatus of the present invention are configured to include biocompatible materials that meet ISO 10993 standards for long-term implants, and/or are able to withstand, without wear, long term normal ranges of physiological loading (i.e., over the lifetime of the implant, or up to about 40×106 cycles) of between about 1250 Newtons (N) (280 lbf) and 2250 N (500 lbf) axial compression; 100 N (25 lbf) and 450 N (100 lbf) of both lateral and sagittal shear, respectively, through full ROM. Additionally, the prosthetic nucleus apparatus of the present invention are preferably able to tolerate short term (e.g., over about 20 continuous cycles) maximum physiological loads through full ROM of about 8000 Newtons (N) (1800 lbf) axial compression; about 2000 N (450 lbf) lateral shear; and about 3000 N (675 lbf) sagittal shear, without failing.

In the context of the present disclosure, the term “binary” refers to prosthetic nucleus apparatus which are configured as an assembly including a barrier-sealant membrane and bulk prosthetic nucleus material.

In the context of the present disclosure, “bulk” typically refers to the fact that the prosthetic nucleus material is larger by volume than the first component, as the barrier sealant membrane is generally dispensed to form a relatively thin layer, or layers.

In the context of the present disclosure, the term “cure” applies to partial or complete curing and refers to a change from a first state, condition, and/or structure in a material, such as a curable polymer or hydrogel, generally by means of a change in cross-linking triggered by means of application of one or more variables, such as a change in pH or temperature; exposure to a curing catalyst or to radiation; passage of time, or the like, to a second altered state.

It will be understood that, in the context of the binary prosthetic nucleus apparatus, it is preferred that triggers and/or in situ curing processes and agents used in forming components are selected based on an absence of resulting deleterious effects.

It will be further understood that the binary prosthetic nucleus apparatus may include a barrier sealant membrane that may conformably contact and/or affix to the surfaces of the intervertebral disc space through a curing process which can comprise evaporation in situ cross-linking. Cross-linking by evaporation can create a polymer film that has material properties inherently dependent on the thickness of the film. Thicker films generally taking longer to dry and having a higher degree of crystallinity than thinner films of the same composition.

It will be further understood that as used in the present disclosure, “component materials” refer to one or a plurality of synthetic or natural hydrogels or blends or hybrid hydrogels e.g., with elastomers; biopolymers; protein polymers; or any combinations thereof, which are biocompatible materials selected as suitable for delivery and use in vivo according to their intended function (e.g., sealant; tissue-repair; barrier; membrane; prosthetic nucleus pulposus), and with requisite biomechanical moduli and physical properties (e.g., elasticity; cold flow; viscosity; solubility; permeability; degradability, etc.) under physiologic conditions.

As used herein, the term “biocompatible” refers to an absence of chronic inflammation response or cytotoxicity when or if physiological tissues are in contact with, or exposed to (e.g., wear debris) the materials and apparatus in accordance with the present inventions.

As will be discussed below, in other aspects of the invention, the barrier sealant membrane include formulations and methods to regulate or enhance solubility, permeability, mechanical bonding between components, and tissue cohesion, to enhance its function in serving as a tissue sealant; as a selectively permeable membrane or barrier; as a repository for drug delivery or therapies for tissue repair. For example, in one aspect of the invention, a barrier sealant membrane may be configured with component materials which include biopolymers networks or proteins which enhance both tissue sealing and repair. In yet another aspect, tissue sealing is achieved by chemical cross-linking with substantially reduced or no accompanying necroses.

Methods and apparatus for dispensing the binary prosthetic nucleus apparatus, and for facilitating formation and/or in situ curing of its components are also disclosed. In particular, the barrier sealant membrane component materials dispensed by means and in the manner as disclosed herein result in the in vivo formation and in situ cure of an effective barrier or delivery system for therapeutic treatment of tissue surfaces in the interior disc space, including disuse sealants that will cohesively adhere to the surface to which it is applied. Suitable component materials systems are disclosed, along with methods for making the barriers that are compliant and capable of conforming to the three dimensional tissue structures within the interior of the intervertebral disc space, and able to withstand, transfer and distribute loads and stresses associated with mobility of the spine during and subsequent to therapy.

### BRIEF DESCRIPTION OF THE DRAWINGS

FIG. 1 illustrates a cross-sectional side view of an embodiment of a prosthetic nucleus apparatus in accordance with the present invention positioned within a spinal motion segment;

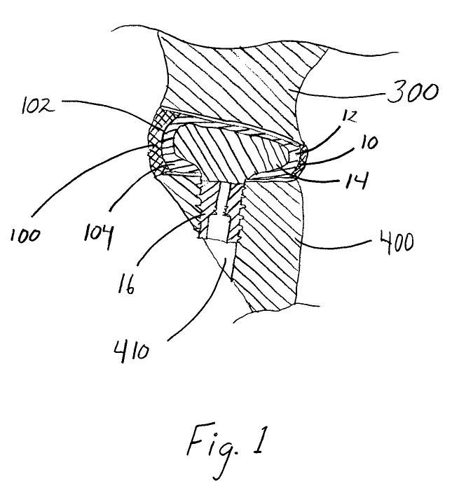

FIG. 2 illustrates a cross-sectional front view of an embodiment of a prosthetic nucleus apparatus in accordance with the present invention positioned within a spinal motion segment;

FIG. 3 illustrates a cross-sectional top view of an embodiment of a prosthetic nucleus apparatus in accordance with the present invention positioned within a spinal motion segment;

FIG. 4 illustrates a side view of an embodiment of a deploying apparatus having a dual chamber configuration for mixing and deploying components of the barrier sealant membrane and/or the prosthetic nucleus material of embodiments of a prosthetic nucleus apparatus in accordance with the present invention;

FIG. 5 illustrates a side view of an embodiment of a deploying apparatus having a single chamber for deploying components of the barrier sealant membrane and/or the prosthetic nucleus material of an embodiment of a prosthetic nucleus apparatus in accordance with the present invention; and

FIGS. 6A, 6B and 6C illustrate embodiments of nozzles for deploying the components of the barrier sealant membrane and/or the prosthetic nucleus material within a de-nucleated space of an intervertebral disc; and

### DETAILED DESCRIPTION OF THE INVENTION

Embodiments of prosthetic nucleus apparatus 10 and delivery apparatus 210 and their components for introduction of a prosthetic nucleus apparatus 10 are generally illustrated throughout the figures. A prosthetic nucleus apparatus 10 in accordance with the present invention is configured to be positioned within a de-nucleuated space 104 within an intervertebral disc 100. In one aspect, the prosthetic nucleus apparatus 10 is configured to at least in part replace at least one function of the native nucleus pulposus. Prosthetic nucleus apparatus 10 generally includes a barrier sealant membrane 12 and a prosthetic nucleus material 14. In an aspect of the present invention, a plug 16 may also be provided. The prosthetic nucleus apparatus 10 is positioned within the de-nucleated space 104 and will typically exert a force against the vertebral end plate of superior vertebral body 300 and a vertebral end plate of the inferior vertebral body 400 adjacent to the intervertebral disc 100 in which the prosthetic nucleus apparatus is implanted. The plug 16 may be inserted into an axial bore 410 in the inferior vertebral body 400, or other point of introduction, after or before the introduction of the barrier sealant membrane 12 and/or the prosthetic nucleus material 14. The plug 16 may also be chemically or mechanically bound to one or more of the barrier sealant membrane 12 and the prosthetic nucleus material 14.

The barrier sealant membrane 12 of the present invention is the component of a prosthetic nucleus apparatus 10 which interacts with the tissue surface 102 which defines the de-nucleated space 104 within an intervertebral disc 100. The barrier sealant membrane may contact, abut, conform to, bond to, or otherwise interact with the tissue surface 102. The barrier sealant membrane 12 is typically composed of implantable materials such as those discussed in greater detail below. Typically, the material of the barrier sealant membrane 12 is selected to permit the transition of the material from a liquid sol to an elastomeric conformable gel or coascervate solid in situ. In one aspect, the barrier sealant membrane 12 may be configured to prevent contain the prosthetic nucleus material within the de-nucleated space 104 and thus, prevent expulsion of prosthetic nucleus materials 14 through fissures or other breaches in the annulus fibrosus of the intervertebral disc 100.

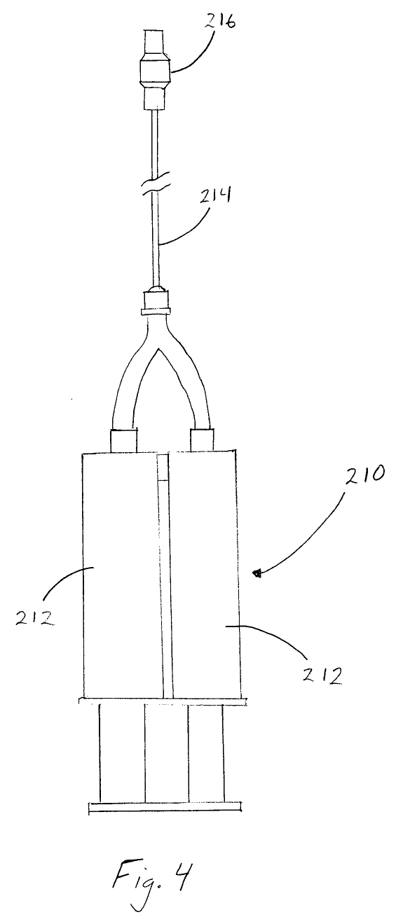

The barrier sealant membrane 12 is typically configured to permit it to be deposited as a film on a tissue surface 102 within a patient. The tissue may be deposited by evaporative coating, spraying, aerosol, atomization, painting, injecting or otherwise onto the tissue as will be recognized by those skilled in the art upon review of the present disclosure. Some exemplary delivery apparatus 210 and their components are generally illustrated in FIGS. 4 to 8A and are discussed in more detail below.

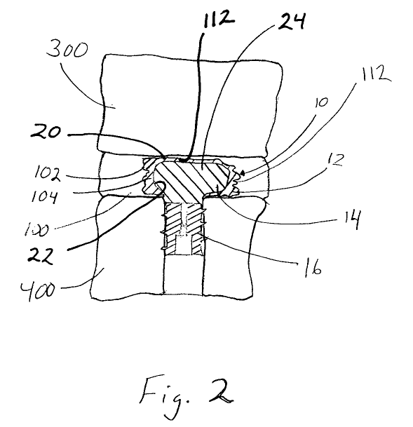

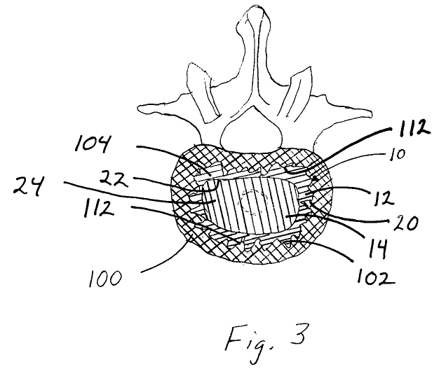

The outer surface 20 of the barrier sealant membrane 12 contacts the tissue surface 102. The tissue surface 102 may include residual tissues from the nucleus pulposus, as well as the tissues of the annulus fibrosus, endplates, and vertebral bodies. The barrier sealant membrane 12 extends over at least a portion of the tissue surface(s) 102. In one aspect, the barrier sealant membrane 12 is configured to conform to the contours of structures 112 defined by the tissue surface 102 that are frequently an artifact of the various de-nucleating procedures, including those disclosed in the documents incorporated by reference herein. Exemplary structures 112 and corresponding shape of the outer surface 20 of barrier sealant membrane 12 which conforms to the shape of the structures 112 are illustrated in both FIG. 2 in a cross-section through the vertical plane and FIG. 3 in a cross-section through the horizontal plane. Those skilled in the art will recognize that structures 112 may take a variety of forms, including both macro and micro level structures, depending on the state of the nucleus as well as the tools, techniques and conditions used for removal of the native nucleus pulposus. In one aspect, the outer surface 20 of the barrier sealant membrane 12 conforming to the structures 112 of the tissue surface 102 can mechanically bond the barrier sealant membrane 12 to the tissue surface 102. In another aspect, the barrier sealant membrane may include a surfactant or, depending on the composition of the barrier sealant membrane 12, additional surfactants at least on the outer surface 20. The surfactant or additional surfactant may reduce the surface tension of the barrier sealant membrane 12, particularly when the barrier sealant membrane 12 is in a liquid form, to permit the barrier sealant membrane 12 to better conform to the structures 112 of the defined by the tissue surface 102. The barrier sealant membrane 12 may also be composed of a material which will enhance the cohesiveness of the barrier sealant membrane 12 to the tissue surface 102, and/or chemically bond the barrier sealant membrane 12 with the tissue surface 102. Alternatively, a cross-linker or conditions may be used to induce the chemical bonding of the barrier sealant membrane 12 to the tissue surface 102. These bonds may include covalent, ionic, and hydrogen bonds as well as Van der Waal's interactions.

An inner surface 22 of the barrier sealant membrane 12 defines a chamber 24 which is configured to at least in part contain the prosthetic nucleus material 14. The chamber may also, in part, be defined by portions of exposed tissue surface 102. The chamber 24 may also be fully or partially enclosed by the plug 16. The chamber 24 is typically sized to receive a desired volume of prosthetic nucleus material 14 to effectively treat the patient. When the de-nucleated space 104 and the chamber 24 are substantially circular in cross-section, the chamber 24 may be formed substantially concentric with the de-nucleated space 104. In one aspect, the chamber 24 may be centrally positioned in three-dimensions within the de-nucleated space 104. The size of the chamber 24 relative to the size of the de-nucleated space 104 is inversely proportional to the amount of material used to form the barrier sealant membrane 12. The inner surface 22 of the barrier sealant membrane 12 may be smooth or irregular in shape. When irregular in shape, the shape may facilitate the mechanical bonding of the prosthetic nucleus material 14 to the inner surface 22 of the barrier sealant membrane 12. The inner surface 22 of the barrier sealant membrane 12 may also be porous. When porous, the prosthetic nucleus material 14 may mechanically interact with the pores to mechanical bond of the prosthetic nucleus material 14 to the inner surface 22 of the barrier sealant membrane 12.

The prosthetic nucleus material 14 is generally positioned within the chamber 24 defined by the barrier sealant membrane 12. The prosthetic nucleus material 12 may generally function to provide support, transfer and/or distribution of compressive loads to physiologic disc structures for the chamber 24 in situ. The prosthetic nucleus material 14 is typically selected to provide the desired biomechanical properties and physical characteristics in view of its volume, shape, location, and purpose to effect the desired treatment of the patient. In addition or in the alternative to the mechanical bonding and interactions and/or cohesion, the prosthetic nucleus material 14 may have a chemical composition which will chemically bond to the barrier sealant membrane 12. Alternatively, a cross-linker or conditions may be used to induce the chemical bonding of the prosthetic nucleus material 14 to the barrier sealant membrane 12. These bonds may include covalent, ionic, and hydrogen bonds as well as Van der Waal's interactions.

In one aspect of the binary prosthetic nucleus apparatus 10, components are configured and component materials are selected according to intended function in vivo. For example, component materials are selected based on biostability and on an ability to regulate configurations' stability under physiological conditions and/or in physiological fluids. More specifically, a barrier sealant membrane 12 may be configured to include a releasable or bioabsorbable therapeutic agent, as will be discussed below. Prosthetic nucleus materials 14 are typically selected to include component elastomeric and/or viscoelastic gels, i.e., materials whose viscoelastic properties (e.g., rheology and compressibility) enable them to perform in a functional manner which is substantially equivalent to the biomechanical functioning of the native nucleus pulposus, and thus it is preferred that the prosthetic nucleus materials 14 be biostable and non-degradable, i.e., to withstand load, resist shear stresses and fatigue forces, or other factors that might otherwise induce fragmentation or otherwise promote extrusion or migration, or fractional mass loss over time.

In one aspect, bulk prosthetic nucleus material 14 may be configured from component materials which include at least one elastomeric material. The Durometer Shore A hardness of the component material may be in the range of substantially about 20-90. Further, the component material, as dispensed, may be stable and biocompatible in vivo, e.g., such as silicone rubber. In one embodiment, the barrier sealant membrane 12 can be configured as a relatively thin and expandable membrane including silicone elastomer which serves as a containment cell for subsequently dispensed prosthetic nucleus material 12. The barrier sealant membrane 12 and the prosthetic nucleus material may be the same component material, i.e., silicone. A suitable silicone may be obtained from Nusil Silicone Technology located in Carpeneria, Calif. In one embodiment, the silicone membrane can exhibit elongation of between about 500% and about 1500%, often about 1000%, and may have a membrane wall thickness of about 0.220″. Following in situ cure and formation, there may remain substantially only one (as visualized fluoroscopically) distinct component. While prosthetic nucleus apparatus 10 configured in this manner can exhibit good biomechanical properties, the barrier sealant membrane 12 component in this embodiment is typically impermeable to the passage of physiologic fluid into and out of the intervertebral disc, and in this respect does not function in the manner of the physiologic intervertebral disc.

A plug 16 may also be provided to seal the point of entry of the materials of the prosthetic nucleus apparatus 10. As generally illustrated in the figures for exemplary purposes, the plug 16 may be particularly configured to preclude leakage or expulsion of prosthetic nucleus material 14 from an axial access bore 410 through the inferior vertebral body 400 leading to the de-nucleated space 104 or other passage for access into the de-nucleated space 24. In one aspect, the plug 16 may be a solid piece of material configured to block and/or seal the point of entry. In another aspect, the plug 16 may define a lumen through which materials of the barrier sealant membrane 12 and prosthetic nucleus materials may be introduced into the lumen. When a lumen is present, the lumen may be configured to limit the ability of the barrier sealant membrane 12 material and/or prosthetic nucleus material 14 to migrate through the channel. In still other embodiments, plug 16 may define a lumen regulated by a unidirectional valve. Any one of numerous valve configurations, e.g., self-sealing valve assemblies or flow-stop devices may be suitable. Typically, the plug 16 is configured to be mechanically secured within the point of introduction. In some exemplary configurations, the plug 16 may be in the form of a smooth rod, threaded rod, a tube, or a threaded tube. As illustrated in the figures for exemplary purposes, the plug 16 may be inserted into the proximal end of the inferior vertebral body 400 of the motion segment of the intervertebral disc 100 into which the prosthetic nucleus apparatus 10 is deployed. The plug 16 is positioned to extend sufficiently through and into the vertebral body to permit it to resist the forces to which the barrier sealant membrane 12 and the prosthetic nucleus material 14 are subjected while retaining its function as a stop flow. Materials suitable as plugs 16, such as non-absorbable threaded plugs, including those fabricated from biocompatible metals, medical grade polyether-ether-ketone (PEEK) such as that commercially available from Invibio Inc., in Lancashire, United Kingdom, or polyether-ketone-ketone (PEKK) available from Coors-Tech Corporation, in Colorado, or alternatively, conventional polymethylmethacrylate (PMMA); ultra high molecular weight polyethylene (UHMWPE), or other suitable polymers in combination with autologous or allograft bone dowels may be used as plugs 16.

The native nucleus pulposus generally consists of type II collagen (cartilage like) and large protein macromolecules called proteoglycans. These native materials absorb water into the intervertebral disc and are extremely important to the biomechanical properties of the intervertebral disc. Thus, the selection of component materials may include materials with the ability to absorb water including materials such as hydrogels and/or viscoelastic gels which can be introduced into the de-nucleated space 104 in aqueous solution, or in dry form (e.g., substantially dehydrated, or particulate), or as microspheres or beads which may then be reconstituted. For example, one hydrogel is formulated as a mixture of hydrogel polyacrylonitrile or any hydrophilic acrylate derivative with a unique multiblock copolymer structure or any other hydrogel material having the ability to imbibe and expel fluids while maintaining its structure under various stresses. As yet another example, the hydrogel can be formulated as a mixture of polyvinyl alcohol (PVA) and water. PVA as a prosthetic nucleus replacement/augmentation material has previously been shown to have no adverse local or systematic tissue reactions, and has been demonstrated to have a compressive modulus greater than 4 MPa and a compressive strength greater than 1 MPa (Bao, Q.-B. and P. A. Higham. Hydrogel Intervertebral Disc Nucleus. U.S. Pat. No. 5,976,186. Nov. 2, 1999). However, bulk prosthetic implants using PVA are not generally considered stable within the physiological environment due to the fact that PVA is a semicrystalline, hydrophilic polymer that can undergo dissolution. The dissolution process is believed to be due to unfolding of PVA crystal chains that join the amorphous region of the polymer, disentangle, and eventually dissolve, and polymer chain dissolution results in a network with a decreased mechanical stiffness resulting from a larger network mesh size. Larger crystals, which undergo a slower dissolution process, are found in semicrystalline PVA hydrogels that have higher PVA molecular weights. Accordingly, in one aspect of the present invention, it is believed that the barrier sealant membrane 12 advantageously substantially limits fractional polymer mass loss, such as that of PVA. In yet another aspect of the invention, in one embodiment, PVA is blended with about 0.5% to about 5.0% by weight of PVP which serves to stabilize bulk prosthetic nucleus material 14. In one aspect of the invention, the hydrogel prosthetic nucleus material 14 is dispensed as formed below the equilibrium level of hydration within a semi-permeable barrier sealant membrane 12 and will swell as it absorbs physiological fluids within the de-nucleated space 104 and fluids that pass through the barrier sealant membrane 12, preferably in the manner of a native nucleus pulposus. As used herein, the term “semi-permeable” refers to barrier sealant membrane 12 which are permeable to the (selectively lateral or bilateral) passage of such fluids but which retain the bulk polymer(s). Moreover, as will be discussed, the degree of permeability of these materials, and rates of hydration may be regulated. In another embodiment, suitable fluids are introduced into the intervertebral disc space through the axial access bore, once the prosthetic nucleus material 14 has been introduced in a substantially dehydrated/de-swollen state. When fully hydrated, the hydrogel prosthetic nucleus material 14 may have a water content of between 25-95%. Natural polysaccharides, such as carboxymethyl cellulose or oxidized regenerated cellulose, natural gum, agar, agrose, sodium alginate, carrageenan, fucoidan, furcellaran, laminaran, hypnea, eucheuma, gum arabic, gum ghatti, gum karaya, gum tragacanth, locust beam gum, arbinoglactan, pectin, amylopectin, gelatin, hydrophilic colloids such as carboxymethyl cellulose gum or alginate gum cross-linked with a polyol such as propylene glycol, and the like, also form hydrogels upon contact with aqueous surroundings. Synthetic hydrogels often exhibit a greater volume expansion and/or rates of expansion. Specifically, synthetic polymeric hydrogels generally swell or expand to a very high degree, usually exhibiting a 2 to 100-fold volume increase upon hydration from a substantially dry or dehydrated state. Synthetic biostable hydrogels appropriate for use in the present invention may include for example, poly(hydroxyalkyl methacrylate), poly(electrolyte complexes), poly(vinylacetate) cross-linked with hydrolysable bonds, and certain N-vinyl lactams. Biostable materials, because they are less susceptible to leaching, may offer advantages with respect to less risk of cytotoxicity.

In a certain aspects, the hydrogel material can include a polyacrylonitrile (PAN) manufactured under the trade name Hypan® by Hymedix International, Inc., and has a water content of about 80-95%. Another hydrogel system includes natural or synthetic hyaluronic acid (HA) or hyaluronan gels or blends that may be chemically altered to enhance structure, e.g., scaffolding ability or physical state, and optimize biomechanical properties in situ via laser exposure, e.g., to convert liquid into a solid. Hyaluronic acid a natural substance that gives structure to tissue, lubricates movable parts, and absorbs shock in joints. Cross-linked hyaluronic acid, such as is available from Fidia Corporation in Italy, is an example of a suitable material, however, many natural and man-made hydrogels or blends thereof may be configured to achieve similar properties without inflammatory response. As will be discussed below, in an exemplary aspect, these are component materials included in barrier sealant membrane 12 to enhance tissue repair. Yet other hydrogels (PEG; PEO; PVP) or blends of hydrogels (e.g., PVA/PVP; PEG-based/PE glycated hydrogels; cross-linked aliphatic polyoxaamide polymers; or combinations of synthetic and native polypeptides or glycosaminoglycans (GAGs) such as such as actin, fibrinogen, collagen and elastin; chondroitin, keratin and dermatan sulfate; chitosan) and/or elastomers or other combinations (e.g., incorporation of an ionic or hydrophobic monomer into the hydrogel network, to engineer a reversibly responsive polymer) that optimize desired intramolecular and intermolecular bonding arrangements and reproduce the viscoelastic properties of the native nucleus pulposus, may also be used. When PVA is included in a blend, PVA may be stabilized by the addition of PVP will typically be used in small percentages by weight such as for example 0.5% to 5.0% to prevent dissolution of the PVA. Conceptually, this is enabled from understanding fundamental relationships between the structure of the polymer (e.g., molecular weight; cross-linking density, etc.) under physiological conditions and the physical properties of the resulting hydrogels. Means for regulating chemical structure and physical properties via reconstitution in vivo or formation via in situ cure are discussed below. For example, as also noted earlier, the prosthetic nucleus apparatus 10 are frequently deployed following complete or partial nucleectomy to remove all or most of the nucleus pulposus which creates a de-nucleated space 104 within the intervertebral disc 100. However, the access tract through which a prosthetic nucleus apparatus containing prosthetic nucleus material 14 is axially deployed will frequently be smaller spatially than the volume of the de-nucleated space 104 to be augmented or replaced. To compensate for the spatial discrepancy, in one aspect of the invention, component materials can be dispensed in a substantially dehydrated condition, for example, using a glycerin carrier. In another aspect, component materials can be dispensed as lyophilized (freeze dried) particulates or powders. Hydration rates will vary depending on the nature functional groups and the surface to volume ratio of the hydrogel. For example, component materials that include charged functional groups, e.g., carboxyl or sulfonic acid groups, that enhance the swellability of hydrogels are more hyperosmotic. Moreover, crushed dried hydrogel beads are expected to swell faster to the equilibrium water content state than a rod shaped implant of comparable volume. Macroporosity or microporosity or surface texture may be created in the hydrogels to increase the surface area for ingress of aqueous fluids, thereby enhancing hydration or control of hydration. Pores formed in the dried hydrogel may create capillary forces that, i.e., a sponge-like effect, to cause rapid absorption of water and concomitant rapid expansion and deployment of the hydrogel. In yet another embodiment, hydration rates are enhanced by making the dried hydrogels hypertonic by the addition of water soluble salts or other agents, including solvents or low molecular weight excipients or oligomers. Such agents rapidly dissolve in an aqueous setting and generate an osmotic driving force that accelerates the hydration process. The hydrogels are typically 3-dimensional structures consisting mainly of hydrophilic (i.e., very high affinity for water) polymeric materials or copolymers which retain water within a substantially insoluble network in which stability that is achieved through the presence of chemical or physical cross-links (e.g., entanglements; crystallites; primary covalent or secondary hydrogen, ionic, or Van der Waals bonds). In this manner, the overall bulk of the prosthetic nucleus apparatus 10 can be reduced, allowing it to be inserted through a smaller access and the subsequent re-hydration, results in an increase in volume of the hydrogel, which is preferably only limited by the volume of the de-nucleated space 104, resulting in uniform conformal contact with the tissue surfaces 102 of the intervertebral disc 100 to distract and restore intervertebral disc height. The resulting prosthetic nucleus apparatus may effectively distribute physiologic loads, e.g., compressive loads, i.e., assume one or more aspects the biomechanical function of the native intervertebral disc. In yet another aspect, precursor macromolecules in aqueous solutions below the equilibrium level of hydration may be dispensed into the de-nucleated space 104 for formation in situ. FIG. 4 generally illustrates a dispensing apparatus 210 capable of dispensing a bulk prosthetic nucleus material 14. In this aspect of the invention, the molecular weight between cross-links may be used as a measure to control the rate of hydration, and hydration initiates the transformation, by causing dissolution of water-soluble components and inducing or regulating nearly simultaneous cross-linking, e.g. by means of polyfunctional groups, such as on biopolymers (e.g., proteins) to form an insoluble network. In yet another aspect, component materials may remain fluid, or by introducing the hydrogel or polymeric material in a first state or condition (e.g., flowable), and allowing or inducing conversion of component materials so as to transform to a second phase or state, e.g., a solid state. In this manner, the material can be introduced through the smallest possible access and yet still be provided in sufficient quantity to fill the de-nucleated space 104 and provide the desired function. Such transformations in component materials may be initiated in responsive to changes in environmental factors, by exhibiting a corresponding change in physical size or shape, so-called “smart” gels. More specifically, component materials including hydrogels modify their molecular arrangement, volume and/or phase when acted upon by a specific stimulus such as temperature, light, a pH change or other chemical inducement, osmotic pressure or mechanical stress, or electric field, and selection of the prosthetic nucleus pulposus material of the present invention is not limited in scope with respect to materials' trigger stimuli, and may include those that are either chemically and/or physically cross-linked gels (e.g., via ion-complexation or those that are thermoreversibly cross-linked) as suitable, except, as previously noted, it is preferred that triggers and/or in situ curing processes and agents used in forming components are selected based on an absence of resulting deleterious effects, such as necroses from the presence of residual cross-linking agents. Examples of methods to convert a material from a first flowable state to a second solid state include but are not limited to: a temperature phase change as from a melted state to a solid to a cascervate or gel state (e.g. thermoreversible between 10 degrees Celsius to about 62 degrees Celsius and often between about 30 degrees Celsius to about 47 degrees Celsius); polymerization of a monomer or low molecular weight polymer such as with the use of a catalyst (e.g., enzymatic); laser or UV cross-linking of a liquid polymer resulting in a solid (e.g., photoinitiated); and leaching of a solvent by replacing it with water (for example: polyacrylonitrile-polyacrylamide hydrogel can be dissolved in dimethylsulfoxide (DMSO) resulting in a flowable liquid which will instantly transform to a solid in the presence of water, into which the DMSO will preferentially flow). More specifically, conversion of a material from a first flowable state to a second solid state includes the use of reverse gelation polymers, such as Pluronic™, commercially available from BASF, Inc., Mount Olive, N.J. (USA), that are liquid at room temperature and form a solid at elevated temperatures such as body temperature, etc. Other thermoreversible hydrogels, such as those formed of amorphous N-substituted acrylamides in water, or natural or genetically engineered elastin like proteins (ELPs) undergo reversible gelation when heated or cooled at certain temperatures (lower critical solution temperature, LCST). Such gels, which are insoluble under physiological conditions, also advantageously may be used for practicing the present invention, particularly to the extent that they are modifiable to assure semi-permeability with respect to the passage of water and physiologic fluids into the de-nucleated space 104 to maintain hydration of and hydrostatic pressure therein.

In one aspect of the present invention, the barrier sealant membrane 12 component materials include thermo-responsive gelation of polymer systems with azo and peroxy functional groups that exhibit thermally labile linkages. For example, in one embodiment, a polymer network including copolymerized poly(ethylene oxide) and poly(l-lactic acid), can be exploited for drug delivery, by means of dispensing via injection through trans-sacral axial access, an aqueous solution of precursors at less than about 45 degrees Celsius that form a gel upon cooling to the physiological temperature of 37 degrees Celsius. In small volumes, this heat differential may be tolerated in vivo without accompanying necrosis or other detrimental effects. In another aspect, the present inventive binary prosthetic nucleus apparatus 10 may utilize enzymes to induce increases in viscosity, e.g., cross-linking or gelation have the advantages of being biocompatible and substantially atraumatic (if the enzyme is not immunogenic), not requiring a chemical initiator, and not resulting in temperature changes at the site. For example, the annulus fibrosus is included of glycosaminoglycans, proteoglycans, and Type II collagen. Biopolymers, such as collagen; glycosamino glycans, or carbohydrates, may be allosterically modified. In one aspect of allosteric modification, transition of barrier sealant membrane 12 component material hydrogel formation and stabilization via in situ cure from a liquid to solid state is triggered enzymatically, e.g., by transglutaminase enzymes, to cross-link biopolymers or proteins in vivo. One advantage of such a trigger may be in the barrier sealant membrane 12 functioning as an in situ delivery vehicle for a range of therapeutic compounds (e.g., capitalizing on biodegradability, through chemical hydrolysis or as enzymatically catalyzed). In yet another aspect of the prosthetic nucleus apparatus 10, the components may include bioactive hydrogels which can be photopolymerized in vivo and/or in vitro in the presence of photoinitiators using visible or ultraviolet (UV) light. Specifically, photopolymerization is used to convert a liquid monomer or macromer to ahydrogel by free radical polymerization in a fast and controllable manner under ambient or physiological conditions. More specifically, in this aspect, in vivo photopolymerization of bulk prosthetic nucleus material 14 component materials include dissolving a photoinitiator in the hydrogel precursor solution, and upon exposure to appropriate light source means introduced via the axial access bore, the precursor solution is converted in situ to form the prosthetic nucleus material 14. In another embodiment of this aspect, a thin film (about 100μ) of hydrogel is formed in situ via absorption on the tissue surfaces by first applying a photoinitiator, e.g., eosin; dispensing the barrier sealant membrane 12 component materials including aqueous precursor solutions with a plurality of reactive groups; and exposing the contact barrier sealant membrane 12/photoinitiator/tissue interface by means of an appropriate light source introduced via the axial access bore. The barrier sealant membrane 12 formed in this manner exhibits enhanced fluid and/or nutrient transport across the membrane. Examples of photopolymerizable macromers include PEG acrylate derivatives PEG methacrylate derivatives, such as PEG diacrylate, methacrylate, and propylene fumarate; cross-linkable polyvinyl alcohol (PVA) derivatives, and modified polysaccharides such as hyaluronic acid derivatives and dextran methacrylate.

In an exemplary embodiment, barrier sealant membrane 12 component materials including photo-cross-linked poly(ethylene oxide) [PEO], or block polypeptide or amino acid hydrogels), may be used to enhance tissue cohesiveness for sealing and repair of tissues within the de-nucleated space 104, by serving as a dimensional matrix that promotes tissue formation. In yet another aspect of the barrier sealant membrane 12, photopolymerized water soluble poly(ethylene glycol) (PEG) diacrylate hydrogels barriers were formed from degradable poly(ethylene glycol-colactic acid) diacrylate macromers as coatings on the tissue surfaces. Similarly, barrier sealant membrane 12 may also be formed from gelation systems including PEG; lactic acid oligomers; and tetraacrylate termini, resulting in in situ formation of thin hydrogel barriers on the interior disc surfaces. In another aspect of the invention, barrier sealant membrane 12 component materials which may be cured in situ using one or more of a pluarilty of triggers (i.e., other than photopolymerization), includes water soluble poly(ethylene glycol) (PEG) configured to serve as means for both a tissue scaffolding, and drug delivery. The intrinsic molecular properties of PEG, e.g., water solubility, resistance to protein adsorption, low immunogenicity, and non-toxicity, facilitate its use as the basis of an in vivo hydrogel. Moreover, covalent incorporation of other synthetic or biological polymers into PEG-based hydrogels can allow for the inclusion of additional desirable physical or biological characteristics. For example, the swelling behavior of an ionic hydrogel system, composed of poly(l-glutamic acid) (PLG) covalently cross-linked to PEG, can be adjusted by the variation of pH, thus altering the ionization of the PLG and resulting in the controlled release of pharmaceuticals. In another aspect, the BSM component materials properties may be adjustably optimized during in situ formation by means of reversible cross linking, e.g., due to disassociation of ionic, secondary bonding, or even covalent bonds, based on susceptibility to agents, such as surfactants, e.g., to modify the tissue surface to enhance cohesion of tissue sealants, and/or functional agents to modify component materials solubility. In the context of the present invention, as used herein the term “surfactant” refers to a surface-active agent which is dispensed or applied to modify (e.g., allosterically, by means of functional group interaction between the surfactant and the tissue protein) the surface of the tissue, to enhance barrier sealant membrane's 12 interfacial cohesion and effective sealing, or, for example to alter dissociation (e.g., solubility). For example, a functionalizing agent may interact with a biopolymer or protein to introduce additional polar groups, such as hydroxyl or carboxylic acid groups, to increase solubility and permeability. The surfactant may be applied to the surface prior to dispensing the barrier sealant membrane 12, or introduced in conjunction with the barrier sealant membrane 12, e.g., by mixing or infusion. Agents known to enhance the inventive barrier sealants of the type disclosed herein include, for example, urea or sulfonated aromatic compounds, and certain block copolymers; biopolymers, or structural proteins, e.g., collagen, fibrinogen, and the like. More specifically, in one embodiment, the barrier sealant membrane 12 component materials properties such as solubility may also be manipulated as is know in the art with respect to hyaluronic acid (HA), or HA-based biomaterials, including viscoelastic gels, wherein solubility is decreased. For example, carboxyl groups on hyaluronic acid may be esterified by alcohols to decrease the solubility of the hyaluronic acid. Such processes are used by various manufacturers of hyaluronic acid products (such as Genzyme Corp., Cambridge, Mass.), and have utility in embodiments wherein barrier sealant membrane 12 include component materials intended for tissue sealing and repair. Similarly, the use of functionalizing agents to modify permeability has utility in this aspect of the invention.

For example, in one aspect of the inventive prosthetic nucleus apparatus 10, the barrier sealant membrane 12 is configured to include hydrogels and form membranes that are not bioabsorbable, e.g., are insoluble, and/or impermeable. More specifically, barrier sealant membrane 12 component materials including non-degradable hydrogels made from poly(vinyl pyrrolidone) and methacrylate which are dispensed to sufficiently swell in situ and form relatively thin barrier sealant membranes which are intended to be biostable, rather than bioabsorbable, and to withstand degradation due to heat or hydrolytic or enzymatic activity. As an example, barrier sealant membrane 12 configured in this manner have utility when there is a need to contain or control the migration from the interior disc space of components from certain tissue sealant systems, for example, sealants that include difunctional cross-linking agents such as glutaraldehyde or diisocyanates, or acid anhydrides, where the presence, leakage or leaching of components of this nature may result in necroses. e.g., due to outgassing or exotherms, and the like. In contrast, in another aspect of the prosthetic nucleus apparatus 10, the barrier sealant membrane's 12 component materials include cross-linked polymeric chains of methoxypoly(ethylene glycol)monomethacrylate. These cross-linked polymeric chains of methoxypoly(ethylene glycol)monomethacrylate can have variable lengths of the polyoxyethylene side chains which typically form membranes which are more soluble and permeable. In general, permeability is typically lower as cross-linking and polymer density increases, although this can be modified by rate of formation in situ in membranes which are relatively thinner. It is also possible to affect the rate of barrier sealant membrane 12 formation in situ, for example, by creating a porous structure within component material during its application, so rate of hydration increase. In another aspect of the invention, porosity of the barrier sealant membrane 12 may be created and pore size controlled. For example, in one embodiment, pores are formed in barrier sealant membrane 12 dispensed by spaying or atomization with accompanying air flow during application. In yet other aspects, membrane pore size and permeability are regulated by the speed of application, and thickness and orientation of the layer, or successive layers of barrier sealant membrane 12 dispensed, as well as by the barrier sealant membrane 12 composition, e.g., structure, cross-linking density, the use of surfactants, or agents that alter polymer viscosity (e.g., peroxides, or by means of thixotropic shear), and the like. More specifically, use of polytetrafluorethylene (PTFE) may enhances the formation of pores of smaller size, by as much as a factor of 10, as compared with barrier sealant membrane 12 conventionally formed in situ, in the absence of the surfactant.

In yet another aspect, porosity is formed in dispensing the component materials, e.g., the co-incorporation of a foaming agent during the formation of the hydrogel may lead to more rapid re-hydration due to the enhanced surface area available for the water front to diffuse into the hydrogel structure. Specifically, the barrier sealant membrane 12 component materials may include additional agents or precursors selected so that, for example, a free radical polymerization is initiated when two components of a redox initiating system are brought together, e.g., agent such sodium bicarbonate, exposed to an acidic environment/in an acidic solution resulting in the release of carbon dioxide as a foaming agent that effervesces during in vivo formation (e.g., polymerization via cross-linking) and in situ cure. More specifically, when such incorporation of a foamed gel is desired, a two component mixture of the precursors to a hydrogel forming system may be selected such that foaming and polymerization to form the hydrogel are initiated when the two fluid channels are mixed.

In an exemplary embodiment, the prosthetic nucleus apparatus 10 can exhibit in situ cure rates which can result in formation times that are short (on the order of seconds or less). This may be optimized by component selection which anticipates or controls fluids, pH and temperature inherent to the environment of the de-nucleated space 104. The components may be selected so as not interfere with the binding, e.g., by cohesive forces; primary or secondary bonds or matrix integration with biopolymers (e.g., protein polymers such as collagen) of the barrier sealant membrane 12 to the tissue surfaces being repaired or sealed (and subsequently, implant properties and performance). Times required for in situ curing or formation may be selected to be short enough to not permit solutions of low viscosity (e.g., precursors, monomers, surfactants or other agents, etc.) to flow away and be cleared from an application site before transformation and solidification occurs. In a preferred aspect, rates of formation in situ may be manipulated, e.g., by techniques as just described, for example, to permit sufficient time to verify placement, uniformity, conformability etc. of the barrier sealant membrane 12 in vivo, such as by means of fluoroscopic visualization, e.g., to allow sufficient time permit revision, for example, removal e.g., by means of irrigation and aspiration, and re-insertion of intervertebral disc treatment or augmentation materials. Thus, in a preferred aspect of the invention, the hydrogels include high contrast means, e.g., agents such as metal ions or iodine, to render them visible (i.e., radio-opaque) upon fluoroscopic inspection. With respect to tissue sealants, as noted earlier, component materials in direct contact with tissues in vivo are selected based on tissue compatibility and non-toxic and non-antigenic properties; absence of deleterious effects resulting from in situ cure or formation (e.g., exothermic heat generation from cross-linking); clinical efficacy with respect to elasticity, tensile strength; adhesion or cohesion and permeability, e.g., in the presence of or with respect to physiologic fluids in the specific environmental of use. As an example, the anatomical structures and environment of the intervertebral disc 100 are essentially avascular. Thus, hydrogel selection for the barrier sealant membrane 12 component materials may include tissue sealants. Such tissue sealant selection may be based on permeability not hemostasis, in contrast to tissue adhesives used in would healing. Additionally, hydrogels may generally exhibit slower degradation rates in this avascular environment, and sealants which are not preferred in other environments may have utility in the environment of the subject invention. In one aspect of the invention, the barrier sealant membrane 12 sealant includes aqueous solutions of synthetic or purified (non-antigenic) biopolymers or proteins, such as collagen or collagen-albumin mixtures or slurries; or fibrinogen, thrombin, and the like, or combinations thereof, of suitably highly fibrous; highly cross-linked; high density of solids (e.g., >65 mg/ml). In one embodiment, it is preferred that the biopolymer protein system be modified to be insoluble, and that proteins be of Type 1 when possible and appropriate. In another embodiment, the sealant additionally includes a cross-linking agent, e.g., gluteraldehyde/aldehyde, or other suitable functional groups modified to minimize toxicity and/or necroses. In a preferred aspect, the cross-linking agent(s) include(s) functionalities which reduce residuals or which are materials that are naturally metabolized. In one embodiment, the cross-lintking agent includes at least one citric acid derivative and synthetic or highly purified biopolymer or protein, such as systems as just described, (e.g., collagen; collagen-albumen; collagen; elastin, etc). In a preferred aspect, the cross-linker is a relatively low weight macromolecule including polar functional groups, such as carboxyl groups or hydroxyl groups, that are modified by means of electron attracting groups, e.g., succinimidyl groups.

In yet another embodiment, the barrier sealant membrane 12 tissue sealants and/or barriers (e.g., thicker layers) include hydrocolloids. More specifically, the barrier sealant membrane 12 may be configured to include water soluble hydrophilic colloidal components, e.g., carboxymethylcellulose, in combination with elastomers or biopolymers as sealants or tissue repair matrices, respectively, and wherein the barrier membrane includes non-degradable, semi-permeable film, In other embodiments, barriers may be pectin-based or foam. In one aspect, hydrogels may be selectively combined and partially or completely cured in situ as a flexible substrate or with a biopolymer matix by application of an appropriate variable, to form a membrane or “skin” in vivo on or within a tissue to seal or repair it. Polyvinyl pyrrolidone (PVP) and polyethylene glycol (PEG) are typical examples of hydrogel polymers that may be modified to form hydrophilic polymer films, e.g., by UV cross-linking derivatives of poly vinyl pyrrolidone. In yet another aspect of the invention, binary components' materials are configured for dispensing and subsequent formation such that aqueous solutions include precursor macromers which are self-assembling hydrogels, i.e., they orient into dimensional networks by means of secondary forces versus covalent bonds. In one embodiment, the self-assembling systems form in vivo as tissue sealants. In yet another aspect of the present invention, the hydrogel-based systems include networks formed by self-assembly wherein the morphologies in the barrier sealant membrane 12 copolymer films depend on the film thickness. In another embodiment the dimensional networks formed by means of self-assembling macromers are incorporated into targeted tissue matrices and serve as repair means for fissures or herniations.

In one exemplary embodiment, aqueous solutions include polyethylene glycol, or multifunctional derivatives thereof. In yet another embodiment the aqueous solutions include polyacrylonitrile (PAN), or derivatives thereof. In another embodiment, the aqueous solutions include hyaluronic acid. In another embodiment, the aqueous solutions include a keratin-based hydrogel structural protein matrix, further including hydrophilic cysteic acid groups in the hydrogel. With respective reference to each embodiment noted above, a coagulum (i.e., an insoluble mass or matrix) can be formed by mixing the solutions together, and the coagulum may serve as an effective tissue sealant, as the environment of the interior disc space is relatively dry, for example as compared with wound treatment environments. Moreover, cohesion of the sealant-tissue interface may be subsequently physically reinforced by the presence in vivo (expanded) and insoluble and non-bioabsorbable/non-degradable bulk prosthetic nucleus material 14. The non-degradable binary prosthetic nucleus apparatus 10 formed in this manner may be able to maintain the tissue-sealant interface and to withstand normal physiologic loading without experiencing cold flow; shifting or migration.

In yet another aspect, the barrier sealant membrane 12 component materials including biopolymer systems such as just described above, facilitate repair by means of barrier sealant membrane 12/tissue 104 interactions beyond the surface or interface (e.g., component materials serve as a structural matrix or scaffold). More specifically, in yet another embodiment, the barrier sealant membrane 12 includes biopolymers which are fibrous or filamentous on nature, e.g., of a similar in biomechanical and physical properties to the native connective tissue fibers/structures, and are non-degradable to provide ongoing sealing and/or structural support. In another embodiment, the barrier sealant membrane 12 component systems include a plurality of synthetic and derivatized materials, for example, such as polyethylene glycol (PEG) precursors which are dispensed as aqueous solutions of macromolecules that are mixed together as they are applied in vivo to the targeted tissue surfaces in the de-nucleated space 104 that may to form in situ cured tissue sealants that are compliant and conformable on all surfaces or components with which it interfaces. In yet another embodiment, the components of the PEG polymer system are dispensed as substantially dehydrated materials or powders, or as dried (e.g., lypholized) particulates. In still another embodiment, the component materials may be dispensed, substantially simultaneously, from both aqueous solutions and as substantially dehydrated components.

The prosthetic nucleus apparatus 10 may be configured to not impede the mobility of, and are responsive to the physiological ICOR. In general, the prosthetic nucleus apparatus 10 can preserve or restore mobility by distraction, decompression, and pain relief that enhances patient mobility rather than controlling or managing motion.

In yet another aspect of the present invention includes prosthetic nucleus apparatus 10 which provide motion management, e.g., a semi-constrained range of motion where full range of motion is allowed in combination with increased resistance to motion; or limited range of motion wherein the extent of motion in one or more degrees of freedom is mechanically limited, with or without increased resistance to motion. Prosthetic nucleus apparatus/motion management apparatus devices may be configured to include the barrier sealant membranes and prosthetic nucleus material 14 components and accompanying therapeutic benefits of the present invention, and hence incorporate the mechanical functions of a nuclear replacement or nucleus pulposus material, and in a preferred embodiment, the apparatus is configured as a combination. Specifically, in this aspect, the de-nucleated space 104 is accessed and prepared according the techniques and tools disclosed in commonly assigned U.S. patent application Ser. Nos. 10/971,799; 10/971,781; 10/971,731; 10/972,077; 10/971,765; 10/972,065; 10/971,775; 10/972,299; and 10/971,780 all filed Oct. 22, 2004, and all claiming the benefit of priority from U.S. Provisional Patent Application Ser. No. 60/513,899 filed Oct. 23, 2003, all of the disclosures of which are hereby incorporated by reference in their entirety, and U.S. Provisional Patent Application Nos. 60/558,069 filed Mar. 31, 2004 the disclosure of which is hereby incorporated by reference in its entirety. For example, following access and preparation of a de-nucleated space 104 using the techniques and tools as previously disclosed, the barrier sealant membrane 12 of the present invention can be dispensed into the de-nucleated space 104 according to delivery apparatus 210. Certain of the motion management devices of the type previously disclosed, e.g., a cannulated flex coupler motion management devices including one or more threaded vertebral body anchoring portion(s) and a fenestrated segment in fluid communication with the de-nucleated space 104, may then be deployed into that motion segment, and prosthetic nucleus material 14 dispensed, through the fenestrated segment of the cannulated motion management devices into the de-nucleated space 104 whose interior surfaces are in conformal contact with the barrier sealant membrane 12. In an alternative aspect, the barrier sealant membrane 12 may also be dispensed into the de-nucleated space 104 by dispensing means in fluid communication with the lumen of the motion management devices and through the fenestrated segment.

The prosthetic nucleus apparatus 10 may provide and maintain maximum distraction, while being implantable and functional within a wide range in anatomies. In a certain embodiments, prosthetic nucleus apparatus 10 can provide from between about 2 mm to about 10 mm, of distraction, and can accommodate physiological lateral disc diameter from between about 15 mm up to about 50 mm; sagittal disc diameter from between about 10 mm up to about 40 mm (i.e., in the median plane between the anterior and posterior sides); intervertebral disc heights from between about 5 mm and about 15 mm; and “wedge angles” from between about 5 degrees and about 15 degrees. As used herein, wedge angle refers to the relative angle of the faces of the inferior and superior vertebral endplates of a motion segment, one to the other.

With respect to the method of deploying the inventive binary prosthetic nucleus apparatus, prior to dispensing the barrier sealant membrane 12 and prosthetic nucleus material 14 components, a complete or partial nucleectomy is performed according to the methods and with the instrumentation tools sets disclosed in U.S. patent application Ser. Nos. 10/971,799; 10/971,781; 10/971,731; 10/972,077; 10/971,765; 10/972,065; 10/971,775; 10/972,299; and 10/971,780 all filed Oct. 22, 2004, and all claiming the benefit of priority from U.S. Provisional Patent Application Ser. No. 60/513,899 filed Oct. 23, 2003, to remove all or most of the nucleus pulposus, respectively, to create a de-nucleated space 104 within the intervertebral disc 100. The prosthetic nucleus apparatus 10 of the present invention may then axially deployed into this de-nucleated space 104, again using substantially the same applicable methods and instrumentation described in the above-referenced disclosures.

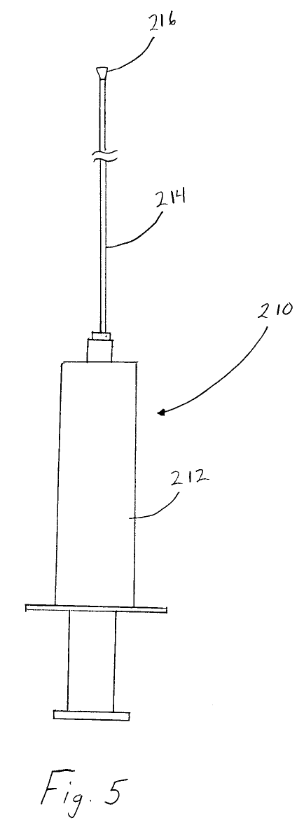

A delivery apparatus 210, including a double barrel syringe assembly as illustrated in FIG. 4 or single barrel syringe assembly as illustrated in FIG. 5, may be provided to mix and/or introduce the various materials and their components as discussed above. The delivery apparatus 210 may be automated or manual in their operation. A deliver apparatus 210 in accordance with the present invention will generally include at least one reservoir 212 and a catheter 214. A nozzle 216 will also typically be provided on a distal end of the catheter 214. Reservoirs 212 are in communication with the catheter 214 to permit the transfer of material from the reservoirs 212 into the catheter 214. Reservoirs 212 are typically capable of being pressurized or are otherwise configured to communicate the material within the reservoir 212 through the catheter 214. The catheter 214 is generally configured to communicate materials from the reservoir 212 to the de-nucleated space 104. The catheter 214 is typically configured to extend from the reservoir 212 to the de-nucleated space 104. The catheter 212 includes an outlet at its distal end. The catheter 212 may be configured to communicate material directly from the outlet into the de-nucleated space 104. In one aspect, the distal end of the catheter 212 may be configured to engage or contact with the plug 16 to facilitate communication of material into the de-nucleated space 104. Nozzles 216 may be provided to deliver, diperse and/or distribute the materials within the de-nucleated space 104. In one aspect, the nozzle 216 may be configured to engage or contact with the plug 16 to facilitate communication of material into the de-nucleated space 104.

As illustrated, each reservoir 212 is in the form of a barrel that may be equipped with a separate plunger 220 to force the material contained therein out through a discharge opening to the catheter 214. As illustrated in FIG. 4 for example, the plungers 220 may be connected to one another at the proximal ends so that a force exerted on the plungers 220 generates equal pressure within each barrel and displaces both plungers 220 an equal distance. If the plungers 220 are not connected, the material components may be delivered separately or in unison.

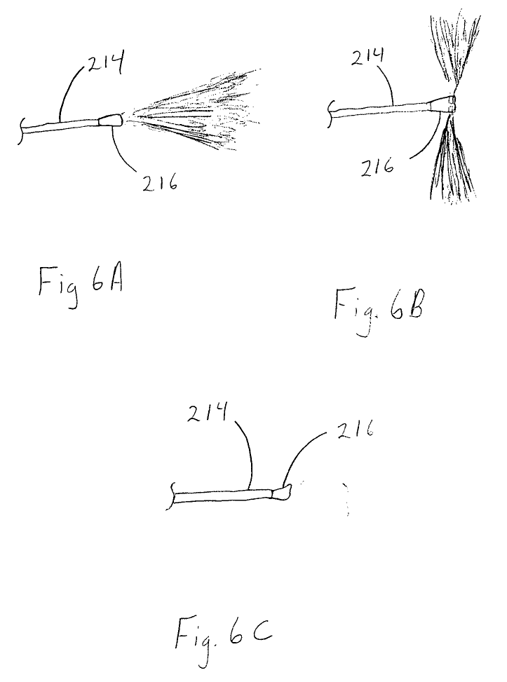

A variety of nozzles 216 are illustrated in FIGS. 6A to 6C at the distal end of catheters 214. Nozzles 216 may spray, sonicate, atomize, direct or otherwise distribute or alter the condition of the material exiting from the distal end of the catheter 212 to coat the tissue surface 102 within the de-nucleated space 104 or to fill or coat the chamber 24. The nozzle 216 may be configured to receive a pressurized fluid such as air to distribute the material in finer particle sizes. Nozzles 216 may be configured to distribute materials in one or more directions from the distal end of the catheter 214. The nozzles 216 may be configured to spray, disburse or distribute material along the longitudinal axis of the catheter 212 as illustrated in FIGS. 6A and 6C or at an angle to the longitudinal axis of the catheter 212. FIG. 6B illustrates nozzle 216 distributing the material in two directions each substantially perpendicular to the longitudinal axis of the catheter 212. In one aspect, the nozzle 216 may be configured as an atomizer or sprayer to dispense a fine mist for direct application coverage.

A programmable syringe pump may be provided as a delivery apparatus 210 to permit automate dispensing of fluid. In one aspect, the programmable syringe pump may be reconfigurable to vary the spray patterns. Generally, parameters of such a delivery apparatus 210 may be set to dispense hydrogel to obtain optimal containment cell dimensions for a particular treatment. When sonicated, liquid hydrogel may be pumped onto a vibrating surface at the tip of the catheter. The vibrating surface of a sonicator is generally used in making fine particles. As shown different vibrating surface tips configurations may be used to vary coatings dimensions and particle sizes. The average particle size atomized or nebulized is related to the surface tension (T), density (p) and the frequency (f) of the liquid. The following formula will help in determining particle size.

$${d}_{h} = 0.73 \sqrt[3]{\frac{T}{ρ {f}_{a}^{2}}}$$

For example, the formula for sonication in the case of water, where T=0.0729 N/m, p=1000 kg/cu. m and f=2.4 mHz, the size of the particles centers around 1.7 microns. The ultrasonic atomizing transducers frequencies typically range from between about 20 kHz to about 120 kHz. In another aspect, a whirl chamber inside the nozzle atomizes fluid without the need for compressed air. The whirl chamber, coupled with a micro-orifice, creates a very fine mist with extremely small droplet sizes of less than 30 microns each.

Methods and apparatus of forming in situ tissue adherent barrier sealant membrane's 12 may be implemented using a delivery apparatus 210 capable of applying two or more viscous cross-linkable components to tissue. In one aspect of the invention, a dispenser includes a plurality of spray nozzles for each of two or more cross-linkable components, and may be co-configured to provide an accompanying vapor flow. Hydrogel(s) components stored in separate compartments are advanced under pressure, e.g., by syringe injection, to the spray nozzles. In one embodiment, the atomized droplets or particulates are dispensed the presence of vapor flow, such as for example, by spraying, and are atomized and mixed to enable subsequent in situ formation, e.g., by cross-linking. The inventive methods and apparatus are suitable for the multi-component hydrogel systems described herein. In a preferred aspect, methods and apparatus for dispensing component materials for in vivo formation and in situ cure are configured to deliver semi-permeable membranes, wherein the barrier sealant membrane 12 has a permeability/porosity that is sufficient to confine the prosthetic nucleus material 14 contained therein within the de-nucleated space 104, while allowing passage (e.g., bi-laterally, into and out of the intervertebral disc) of low molecular weight hydration fluids or therapeutic agents. Preferably, the openings have an average diameter of about 10 micrometers, although other dimensions are acceptable depending on the degree of cross linking and density of the component materials polymer systems, which will vary accordingly. Moreover, the barrier sealant membrane 12 should be delivered in a manner and at a rate which is compliant, in that it is capable of conforming to the three dimensional structure of a tissue surface as the tissue bends and deforms during and after its formation and in situ cure e.g., during the time of therapy. More specifically, the barrier sealant membrane 12 should be sufficiently compliant to both allow and withstand the expansion and contraction of the prosthetic nucleus material 14, such as for example a hydrogel, in a controlled fashion while maintaining conformal contact/sealing engagement with the annulus fibrosus and disc endplates as physiologic loads are transferred by means of hydrostatic pressure to those structures by the bulk nucleus pulposus material. In general, the barrier sealant membrane 12 has a burst strength that is greater than the swelling pressure of the hydrogel core when fully hydrated to prevent rending and loss of the hydrogel core. The ultimate volume of the prosthetic nucleus material 14 within the barrier sealant membrane 12 is typically limited by contact with the superior and inferior vertebral endplates and the annulus fibrosus, preventing disruption of the barrier sealant membrane 12 due to over inflation. By having a barrier sealant membrane 12 that is both flexible and semi-compliant, i.e., the elasticity being “modulus matched” to the native nucleus pulposus, the filled barrier sealant membrane 12 effectively contributes as a dampener. In another aspect, the semi-permeable barrier sealant membrane 12 may be dispensed to include a biaxially oriented membrane configuration. Delivery may include dispensing one or a plurality of layers, to achieve optimum membrane thickness. In one embodiment, the barrier sealant membrane 12 is configured so that that may be modified to be microporous by mechanical means, such as of laser drilling, or by chemical means, e.g., leaching out sacrificial salt particles to achieve a satisfactory end configuration. In an exemplary embodiment, component materials may be delivered by means of directional control to targeted tissue surfaces or cavities, and particle sizes of between about 5 and 30 microns may be formed and particle size may be regulated as a function, for example tip diameter; catheter length; and component material(s) viscosity. In another embodiment, component materials systems may include hydrogel films may be pre-prepared, e.g., lyophilized to remove all water, and then ground or powdered to an appropriate particulate size for dispensing, and subsequent in situ aqueous dissolution and formation. In yet another embodiment, a tissue surfactant agent includes a photoinitiator, applied in sequence or simultaneously with a polymerizable macromer solution to the interior disc surfaces with such that subsequent irradiation in situ results in formation of a cohesive tissue sealant. Surface treatment to increase hydrophilicity may also reduce bacterial adhesion, and the hydrogel polymers and films are softer and more compliant and surface conformable after water absorption, i.e., providing a softer surface for tissue contact, possibly reducing the stimulation of a foreign surface to living tissues and possibly increasing the biocompatibility of the prosthetic nucleus apparatus. Moreover, the adhesion of microorganisms on hydrogel-treated surfaces is reduced because of increased hydrophilicity. In addition the hydrophilicity of the polymer matrices can be crucial to the release of anti-infective agents, and barrier sealant membranes 12 including hydrophilic functional groups can allow water molecules to diffuse easily into the biopolymer matrix and to diffuse out, when exposed to body fluids, with matrix entrapped therapeutic agents, e.g., anti-infective agents, into the surrounding tissue. In another aspect of the inventive prosthetic nucleus apparatus 10, in situ formation of tissue sealant layer or layers of desired thickeness (e.g., application to form multiple or successive layers), may be selectively dispensed, resulting in effective barrier formation and/or effective barriers.

The barrier sealant membrane 12 and the prosthetic nucleus material 14 of the prosthetic nucleus apparatus 10 are typically sequentially introduced into the de-nucleated space 104, in the order and manner as previously described. FIGS. 4 to 6B, discussed in detail above, illustrate delivery apparatus 210 and their component parts that may be used to introduce the barrier sealant membrane 12 and the prosthetic nucleus material 14. More specifically, in one aspect of the invention, component materials are introduced into the de-nucleated space 104 to seal, repair, augment or replace native intervertebral disc tissues using a delivery apparatus 210 is configured to pass through the dilator sheath following the creation of a bore 410 to the intervertebral disc 100 (using bone dilation, drilling, or a combination of the two). In this aspect, components of such a delivery apparatus 210 could be made out of several materials including nitinol tubing, or a flexible plastic material such as polyethylene. Alternatively, the components of delivery apparatus 210 could also be engaged to the proximal end of a fenestrated axial rod, in the same manner and similarly configured as a paste inserter, to deploy nucleus pulposus augmentation or replacement substances to bolster the internal lamina of the annulus fibrosus; distract the intervertebral disc space; and/or distribute or share physiologic loads. The apparatus would have to fit within the dilator sheath (Sheath ID=0.397″) and be at least 10″ in length to track from outside the body to the site of application. The ID of the delivery apparatus needs to be large enough to allow insertion of the prosthetic nucleus material 14 which may vary in viscosity depending on needs re biomechanical properties; volume and nature of material. For example, the cannula of the prosthetic nucleus inserter should be of a sufficient cannula ID so no undue shear forces on the prosthetic nucleus material 14 so as to cause fragmentation or otherwise deleteriously alter biomechanical properties and won't require deleterious or excessive pressure for injection. More specifically, this would be a design consideration for prosthetic nucleus apparatus 10 wherein the prosthetic nucleus material 14 includes a hydrogel infused into the intervertebral disc space (with or without barrier sealant membrane 12) using the disclosed augmentation prosthetic nucleus media inserters. In a preferred aspect, the cannula are provided with interchangeable inserted tips on the distal ends of the dispensing apparatus which accommodate the specific viscosites and physical dimensions of component materials systems and accompanying modifying agents, surfactants, buffers and the like. In an alternative embodiment the distal ends are configured with engagement means when the barrier sealant membrane 12 and prosthetic nucleus material 14 are dispensed as sub-assembly means, i.e., through fenestrations included in a motion management apparatus, as opposed to their direct delivery into the intervertebral disc space. As noted previously, following deployment of the binary prosthetic nucleus apparatus components the axial access bore through which they were dispensed is preferably sealed, e.g., near the proximal end of the vertebral body which is inferior to (each) intervertebral disc(s) into which the component materials were dispensed, by sealing means and materials as previously described.

In another aspect of the present invention the hydrogel sealant may include two or more components sprayed as droplets or particulates separately and simultaneously infused such that in situ formation of the barrier sealant membrane 12 occurs when these components are combined, using a flexibly positioned and directional sealant delivery apparatus 210 onto the target site and wherein the two parts mix and form a hydrogel product consisting mainly of water. In yet another embodiment the sealant may include a plurality of components that are premixed and then dispensed in vivo for selective in situ curing.

Yet another aspect of the present invention includes mixing of a plurality of self-activating components at the time of delivery apparatus 210 including multiple metered cells (e.g., syringes) that may contain volumes of the components that are different or the same. In one embodiment, the delivery apparatus 210 can include with two identical disposable syringes that are joined by a common plunger 220 to assure that the two components are dispensed in equal volumes, during delivery. Such units are commercially available. In another embodiment, the delivery of components, e.g., barrier sealant membrane 12 components in this manner may be dispensing through a delivery apparatus 210 (e.g., atomizer; nebulizer, syringe, etc.) in amounts to evenly mix and form at physiologic temperatures films of sufficient uniformity, thickness, compliancy and conformability and needed to seal or repair fissures, e.g., in the annulus fibrosus.

In yet another aspect of the present invention, kits are provided which include all of the components, mixing materials necessary for preparing barrier sealant membrane 12 and prosthetic nucleus material 14 of the binary prosthetic nucleus apparatus 10 and dispensing them via delivery apparatus 210 for deployment via the trans-sacral axial access bore 410 to the de-nucleated space 104.

While the present invention has been illustrated and described with particularity in terms of preferred embodiments, it should be understood that no limitation of the scope of the invention is intended thereby. For example, features of any of the foregoing methods, and exemplary apparatus shown and briefly described below, may be substituted or added into the others, as will be apparent to those of skill in the art. The scope of the invention is in no way intended to be limited by the brevity or exemplary nature of the material below, and may be further understood from the accompanying Figures. It should also be understood that variations of the particular embodiments described herein incorporating the principles of the present invention will occur to those of ordinary skill in the art and yet be within the scope of the materials described and shown herein.

What is claimed is:

1. An axially implantable apparatus for treating an at least partially de-nucleated intervertebral disc, comprising:
a barrier sealant membrane adapted to abut a tissue surface defining a de-nucleated space within an intervertebral disc, the barrier sealant membrane defining a chamber, wherein the barrier sealant membrane is introduced into the de-nucleated space through an axial bore through an inferior vertebral body; and
a prosthetic nucleus material disposed within the chamber of the barrier sealant membrane, wherein the prosthetic nucleus material is introduced into the chamber through the axial bore through the inferior vertebral body.

2. An axially implantable apparatus, as in claim 1, wherein said barrier sealant membrane and prosthetic nucleus material comprise materials which are insoluble and non-degradable.

3. An axially implantable apparatus, as in claim 1, wherein said barrier sealant membrane comprises materials which are bioabsorbable.

4. An axially implantable apparatus, as in claim 1, wherein said barrier sealant membrane comprises materials which are non-degradable.

5. An axially implantable apparatus, as in claim 1, wherein said prosthetic nucleus material comprises materials which are insoluble and non-degradable.

6. An axially implantable apparatus, as in claim 1, wherein said barrier sealant membrane and said prosthetic nucleus material comprise the same materials.

7. An axially implantable apparatus, as in claim 1, wherein said barrier sealant membrane comprises a material which is bonded to the tissue surface.

8. An axially implantable apparatus implantable apparatus, as in claim 7, wherein said barrier sealant membrane is formed in vivo by means of an in situ cure.

9. An axially implantable apparatus, as in claim 1, wherein the barrier sealant membrane comprises a hydrogel.

10. An axially implantable apparatus, as in claim 9, wherein said hydrogel comprises polyvinyl alcohol and polyvinyl pyrrolidone.

11. An axially implantable apparatus, as in claim 9, wherein said hydrogel comprises polyethylene glycol.

12. An axially implantable apparatus, as in claim 9, wherein said hydrogel comprises polyvinyl pyrrolidone.

13. An axially implantable apparatus, as in claim 1, wherein said barrier sealant membrane comprises an elastomeric material.

14. An axially implantable apparatus, as in claim 13, wherein said elastomeric material comprises a silicone.

15. An axially implantable apparatus, as in claim 13, wherein said elastomeric material comprises a polyurethane.

16. An axially implantable apparatus, as in claim 1, wherein said barrier sealant membrane comprises a blend of an elastomeric material and a hydrogel.

17. An axially implantable apparatus, as in claim 1, wherein said prosthetic nucleus material comprises an elastomeric material.

18. An axially implantable apparatus, as in claim 17, wherein said elastomeric material comprises a silicone.

19. An axially implantable apparatus, as in claim 17, wherein said elastomeric material comprises a polyurethane.

20. An axially implantable apparatus, as in claim 1, wherein said prosthetic nucleus material comprises a hydrogel.

21. An axially implantable apparatus, as in claim 20, wherein said hydrogel comprises polyvinyl alcohol and polyvinyl pyrrolidone.

22. An axially implantable apparatus, as in claim 20, wherein said hydrogel comprises polyethylene glycol.

23. An axially implantable apparatus, as in claim 20, wherein said hydrogel comprises polyvinyl pyrrolidone.

24. A prosthetic nucleus apparatus for treating an at least partially de-nucleated intervertebral disc, comprising:
a barrier sealant membrane adapted to bond to a tissue surface defining a de-nucleated space within an intervertebral disc, the barrier sealant membrane defining a chamber; and
a prosthetic nucleus material positioned within the chamber of the barrier sealant membrane.

25. A prosthetic nucleus apparatus, as in claim 24, further comprising the barrier membrane including a surfactant at least on the outer surface of the barrier sealant membrane to contact the tissue surface and to bond the barrier sealant membrane to the tissue surface.

26. A prosthetic nucleus apparatus, as in claim 24, further comprising the barrier membrane including an agent at least on the outer surface of the barrier sealant membrane to contact the tissue surface and to bond the barrier sealant membrane to the tissue surface.

27. A prosthetic nucleus apparatus, as in claim 26, the agent comprising urea.

28. A prosthetic nucleus apparatus, as in claim 26, the agent comprising sulfonated aromatic compounds.

29. A prosthetic nucleus apparatus, as in claim 26, the agent comprising collagen.

30. A prosthetic nucleus apparatus, as in claim 26, the agent comprising fibrinogen.

31. A prosthetic nucleus apparatus, as in claim 25, further comprising, the barrier sealant membrane and prosthetic nucleus material comprise materials which are insoluble and non-degradable.

32. A prosthetic nucleus apparatus, as in claim 25, wherein said barrier sealant membrane comprises materials which are bioabsorbable.

33. A prosthetic nucleus apparatus, as in claim 25, wherein said barrier sealant membrane comprises materials which are non-degradable.

34. A prosthetic nucleus apparatus, as in claim 25, wherein said prosthetic nucleus material comprises materials which are insoluble and non-degradable.

35. A prosthetic nucleus apparatus, as in claim 25, wherein said barrier sealant membrane and said prosthetic nucleus material comprise the same materials.

36. A prosthetic nucleus apparatus, as in claim 25, wherein said barrier sealant membrane and said prosthetic nucleus material comprise silicone.

37. A prosthetic nucleus apparatus implantable apparatus, as in claim 25, wherein said barrier sealant membrane is formed in vivo by means of an in situ cure.

38. A prosthetic nucleus apparatus implantable apparatus, as in claim 25, wherein said barrier sealant membrane is deposited within the de-nucleated space through an axial bore through one or more inferior vertebrae.

39. A prosthetic nucleus apparatus, as in claim 25, wherein the barrier sealant membrane comprises a hydrogel.

40. A prosthetic nucleus apparatus, as in claim 39, wherein said hydrogel comprises polyvinyl alcohol and polyvinyl pyrrolidone.

41. A prosthetic nucleus apparatus, as in claim 39, wherein said hydrogel comprises polyethylene glycol.

42. A prosthetic nucleus apparatus, as in claim 39, wherein said hydrogel comprises polyvinyl pyrrolidone.

43. A prosthetic nucleus apparatus, as in claim 25, wherein said barrier sealant membrane comprises an elastomeric material.

44. A prosthetic nucleus apparatus, as in claim 43, wherein said elastomeric material comprises a silicone.

45. A prosthetic nucleus apparatus, as in claim 43, wherein said elastomeric material comprises a Polyurethane.

46. A prosthetic nucleus apparatus, as in claim 25, wherein said barrier sealant membrane comprises a blend of an elastomeric material and a hydrogel.

47. A prosthetic nucleus apparatus, as in claim 25, wherein said prosthetic nucleus material comprises an elastomeric material.

48. A prosthetic nucleus apparatus, as in claim 47, wherein said elastomeric material comprises a silicone.

49. A prosthetic nucleus apparatus, as in claim 47, wherein said elastomeric material comprises a polyurethane.

50. A prosthetic nucleus apparatus, as in claim 25, wherein said prosthetic nucleus material comprises a hydrogel.

51. A prosthetic nucleus apparatus, as in claim 50, wherein said hydrogel comprises polyvinyl alcohol and polyvinyl pyrrolidone.

52. A prosthetic nucleus apparatus, as in claim 50, wherein said hydrogel comprises polyethylene glycol.

53. A prosthetic nucleus apparatus, as in claim 50, wherein said hydrogel comprises polyvinyl pyrrolidone.

54. A prosthetic nucleus apparatus for treating an at least partially de-nucleated intervertebral disc, comprising:
a barrier sealant membrane adapted to contact a tissue surface defining a de-nucleated space within an intervertebral disc; and
a prosthetic nucleus material positioned within a chamber defined by the barrier sealant membrane, the prosthetic nucleus material bonded to the barrier sealant membrane.

55. A prosthetic nucleus apparatus, as in claim 54, further comprising the barrier membrane adapted to cohere to a tissue surface and including a surfactant at least on an outer surface of the barrier sealant membrane to contact the tissue surface and to bond the barrier sealant membrane to the tissue surface.

56. A prosthetic nucleus apparatus, as in claim 54, wherein the barrier sealant membrane and prosthetic nucleus material comprise materials which are insoluble and non-degradable.

57. A prosthetic nucleus apparatus, as in claim 54, wherein said barrier sealant membrane comprises materials which are bioabsorbable.

58. A prosthetic nucleus apparatus, as in claim 54, wherein said barrier sealant membrane comprises materials which are non-degradable.

59. A prosthetic nucleus apparatus, as in claim 54, wherein said prosthetic nucleus material comprises materials which are insoluble and non-degradable.

60. A prosthetic nucleus apparatus, as in claim 54, wherein said barrier sealant membrane and said prosthetic nucleus material comprise silicone.

61. A prosthetic nucleus apparatus, as in claim 54, wherein said barrier sealant membrane comprises a material which is bonded to the tissue surface.

62. A prosthetic nucleus apparatus implantable apparatus, as in claim 54, wherein said barrier sealant membrane is formed in vivo by means of an in situ cure.

63. A prosthetic nucleus apparatus implantable apparatus, as in claim 62, wherein said barrier sealant membrane is deposited within the de-nucleated space through an axial bore through one or more inferior vertebrae.

64. A prosthetic nucleus apparatus, as in claim 54, wherein the barrier sealant membrane comprises a hydrogel.

65. A prosthetic nucleus apparatus, as in claim 64, wherein said hydrogel comprises polyvinyl alcohol and polyvinyl pyrrolidone.

66. A prosthetic nucleus apparatus, as in claim 64, wherein said hydrogel comprises polyethylene glycol.

67. A prosthetic nucleus apparatus, as in claim 64, wherein said hydrogel comprises polyvinyl pyrrolidone.

68. A prosthetic nucleus apparatus, as in claim 54, wherein said barrier sealant membrane comprises an elastomeric material.

69. A prosthetic nucleus apparatus, as in claim 68, wherein said elastomeric material comprises a silicone.

70. A prosthetic nucleus apparatus, as in claim 68, wherein said elastomeric material comprises a Polyurethane.

71. A prosthetic nucleus apparatus, as in claim 54, wherein said barrier sealant membrane comprises a blend of an elastomeric material and a hydrogel.

72. A prosthetic nucleus apparatus, as in claim 54, wherein said prosthetic nucleus material comprises an elastomeric material.

73. A prosthetic nucleus apparatus, as in claim 72, wherein said elastomeric material comprises a silicone.

74. A prosthetic nucleus apparatus, as in claim 72, wherein said elastomeric material comprises a polyurethane.

75. A prosthetic nucleus apparatus, as in claim 54, wherein said prosthetic nucleus material comprises a hydrogel.

76. A prosthetic nucleus apparatus, as in claim 75, wherein said hydrogel comprises polyvinyl alcohol and polyvinyl pyrrolidone.

77. A prosthetic nucleus apparatus, as in claim 75, wherein said hydrogel comprises polyethylene glycol.

78. A prosthetic nucleus apparatus, as in claim 75, wherein said hydrogel comprises polyvinyl pyrrolidone.

79. A prosthetic nucleus apparatus for treating an at least partially de-nucleated intervertebral disc formed by a process, comprising:
coating a barrier sealant membrane in a liquid form onto a tissue surface defining a de-nucleated space within an intervertebral disc, the barrier sealant membrane conforming with the tissue surface and defining a chamber; and
injecting a prosthetic nucleus material into the chamber defined by the barrier sealant membrane.

80. A prosthetic nucleus apparatus, as in claim 79, further comprising the barrier sealant membrane adapted to bond to a tissue surface and including an agent at least on an outer surface of the barrier sealant membrane to contact the tissue surface and to bond the barrier sealant membrane to the tissue surface.

81. A prosthetic nucleus apparatus, as in claim 79, further comprising, the barrier sealant membrane and prosthetic nucleus material comprise materials which are insoluble and non-degradable.

82. A prosthetic nucleus apparatus, as in claim 79, wherein said barrier sealant membrane comprises materials which are bioabsorbable.

83. A prosthetic nucleus apparatus, as in claim 79, wherein said barrier sealant membrane comprises materials which are non-degradable.

84. A prosthetic nucleus apparatus, as in claim 79, wherein said prosthetic nucleus material comprises materials which are insoluble and non-degradable.

85. A prosthetic nucleus apparatus, as in claim 79, wherein said barrier sealant membrane and said prosthetic nucleus material comprise silicone.

86. A prosthetic nucleus apparatus, as in claim 79, wherein said barrier sealant membrane comprises a material which is bonded to the tissue surface.

87. A prosthetic nucleus apparatus, as in claim 79, wherein said barrier sealant membrane is formed in vivo by means of an in situ cure.

88. A prosthetic nucleus apparatus implantable apparatus, as in claim 79, wherein the coating of barrier sealant membrane is deposited within the de-nucleated space through an axial bore through one or more inferior vertebrae.

89. A prosthetic nucleus apparatus, as in claim 79, wherein the barrier sealant membrane comprises a hydrogel.

90. A prosthetic nucleus apparatus, as in claim 89, wherein said hydrogel comprises polyvinyl alcohol and polyvinyl pyrrolidone.

91. A prosthetic nucleus apparatus, as in claim 89, wherein said hydrogel comprises polyethylene glycol.

92. A prosthetic nucleus apparatus, as in claim 89, wherein said hydrogel comprises polyvinyl pyrrolidone.

93. A prosthetic nucleus apparatus, as in claim 79, wherein said barrier sealant membrane comprises an elastomeric material.

94. A prosthetic nucleus apparatus, as in claim 93, wherein said elastomeric material comprises a silicone.

95. A prosthetic nucleus apparatus, as in claim 93, wherein said elastomeric material comprises a polyurethane.

96. A prosthetic nucleus apparatus, as in claim 79, wherein said barrier sealant membrane comprises a blend of an elastomeric material and a hydrogel.

97. A prosthetic nucleus apparatus, as in claim 79, wherein said prosthetic nucleus material comprises an elastomeric material.

98. A prosthetic nucleus apparatus, as in claim 97, wherein said elastomeric material comprises a silicone.

99. A prosthetic nucleus apparatus, as in claim 97, wherein said elastomeric material comprises a polyurethane.

100. A prosthetic nucleus apparatus, as in claim 79, wherein said prosthetic nucleus material comprises a hydrogel.

101. A prosthetic nucleus apparatus, as in claim 100, wherein said hydrogel comprises polyvinyl alcohol and polyvinyl pyrrolidone.

102. A prosthetic nucleus apparatus, as in claim 100, wherein said hydrogel comprises polyethylene glycol.

103. A prosthetic nucleus apparatus, as in claim 100, wherein said hydrogel comprises polyvinyl pyrrolidone.

104. A method for treating an at least partially de-nucleated intervertebral disc, comprising:
boring a hole axially through a vertebral body using a trans sacral approach to access a nucleus pulposus of an intervertebral disc;
removing at least a portion of the nucleus pulposus to define a de-nucleated space within the intervertebral disc space;
depositing a barrier sealant membrane onto a tissue surface defining the de-nucleated space within an intervertebral disc; and
deploying a prosthetic nucleus material into the chamber defined by the barrier sealant membrane.

105. A method, as in claim 104, further comprising curing the barrier sealant membrane on the tissue surface.

106. A method, as in claim 104, further comprising bonding the barrier sealant membrane to the tissue surface.

107. A method, as in claim 106, further comprising the barrier sealant membrane including an agent to contact the tissue surface and to bond the barrier sealant membrane to the tissue surface.

108. A method, as in claim 104, further comprising, the barrier sealant membrane and prosthetic nucleus material comprise materials which are insoluble and non-degradable.

109. A method, as in claim 104, wherein said barrier sealant membrane comprises materials which are bioabsorbable.

110. A method, as in claim 104, wherein said barrier sealant membrane comprises materials which are non-degradable.

111. A method, as in claim 104, wherein said prosthetic nucleus material comprises materials which are insoluble and non-degradable.

112. A method, as in claim 104, wherein said barrier sealant membrane and said prosthetic nucleus material comprise the same materials.

113. A method, as in claim 104, wherein said barrier sealant membrane and said prosthetic nucleus material comprise silicone.

114. A method, as in claim 104, wherein said barrier sealant membrane is deposited within the de-nucleated space through an axial bore through one or more inferior vertebrae.

115. A method, as in claim 104, wherein the barrier sealant membrane comprises a hydrogel.

116. A method, as in claim 115, wherein said hydrogel comprises polyvinyl alcohol and polyvinyl pyrrolidone.

117. A method, as in claim 115, wherein said hydrogel comprises polyethylene glycol.

118. A method, as in claim 115, wherein said hydrogel comprises polyvinyl pyrrolidone.

119. A method, as in claim 104, wherein said barrier sealant membrane comprises an elastomeric material.

120. A method, as in claim 119, wherein said elastomeric material comprises a silicone.

121. A method, as in claim 119, wherein said elastomeric material comprises a polyurethane.

122. A method, as in claim 104, wherein said barrier sealant membrane comprises a blend of an elastomeric material and a hydrogel.

123. A method, as in claim 104, wherein said prosthetic nucleus material comprises an elastomeric material.

124. A method, as in claim 123, wherein said elastomeric material comprises a silicone.

125. A method, as in claim 123, wherein said elastomeric material comprises a polyurethane.

126. A method, as in claim 104, wherein said prosthetic nucleus material comprises a hydrogel.

127. A method, as in claim 126, wherein said hydrogel comprises polyvinyl alcohol and polyvinyl pyrrolidone.

128. A method, as in claim 126, wherein said hydrogel comprises polyethylene glycol.

129. A method, as in claim 126, wherein said hydrogel comprises polyvinyl pyrrolidone.

130. A prosthetic nucleus apparatus for treating a de-nucleated intervertebral disc, comprising:
a barrier sealant membrane including an outer surface which conforms to structures of the tissue surface defining a de-nucleated space within an intervertebral disc, the barrier sealant membrane defining a chamber; and
a prosthetic nucleus material positioned within the chamber of the barrier sealant membrane.

131. A prosthetic nucleus apparatus, as in claim 130, further comprising the barrier membrane adapted to bond to a tissue surface and including an agent at least on an outer surface of the barrier sealant membrane to contact the tissue surface and to bond the barrier sealant membrane to the tissue surface.

132. A prosthetic nucleus apparatus, as in claim 130, further comprising, the barrier sealant membrane and prosthetic nucleus material comprise materials which are insoluble and non-degradable.

133. A prosthetic nucleus apparatus, as in claim 130, wherein said barrier sealant membrane comprises materials which are bioabsorbable.

134. A prosthetic nucleus apparatus, as in claim 130, wherein said barrier sealant membrane comprises materials which are non-degradable.

135. A prosthetic nucleus apparatus, as in claim 130, wherein said prosthetic nucleus material comprises materials which are insoluble and non-degradable.

136. A prosthetic nucleus apparatus, as in claim 130, wherein said barrier sealant membrane and said prosthetic nucleus material comprise the same materials.

137. A prosthetic nucleus apparatus, as in claim 130, wherein said barrier sealant membrane comprises a material which is bonded to the tissue surface.

138. A prosthetic nucleus apparatus, as in claim 130, wherein said barrier sealant membrane is formed in vivo by means of an in situ cure.

139. A prosthetic nucleus apparatus, as in claim 130, wherein said barrier sealant membrane is deposited within the de-nucleated space through an axial bore through one or more inferior vertebrae.

140. A prosthetic nucleus apparatus, as in claim 130, wherein the barrier sealant membrane comprises a hydrogel.

141. A prosthetic nucleus apparatus, as in claim 140, wherein said hydrogel comprises polyvinyl alcohol and polyvinyl pyrrolidone.

142. A prosthetic nucleus apparatus, as in claim 140, wherein said hydrogel comprises polyethylene glycol.

143. A prosthetic nucleus apparatus, as in claim 140, wherein said hydrogel comprises polyvinyl pyrrolidone.

144. A prosthetic nucleus apparatus, as in claim 130, wherein said barrier sealant membrane comprises an elastomeric material.

145. A prosthetic nucleus apparatus, as in claim 144, wherein said elastomeric material comprises a silicone.

146. A prosthetic nucleus apparatus, as in claim 144, wherein said elastomeric material comprises a Polyurethane.

147. A prosthetic nucleus apparatus, as in claim 130, wherein said barrier sealant membrane comprises a blend of an elastomeric material and a hydrogel.

148. A prosthetic nucleus apparatus, as in claim 130, wherein said prosthetic nucleus material comprises an elastomeric material.

149. A prosthetic nucleus apparatus, as in claim 148, wherein said elastomeric material comprises a silicone.

150. A prosthetic nucleus apparatus, as in claim 148, wherein said elastomeric material comprises a polyurethane.

151. A prosthetic nucleus apparatus, as in claim 130, wherein said prosthetic nucleus material comprises a hydrogel.

152. A prosthetic nucleus apparatus, as in claim 151, wherein said hydrogel comprises polyvinyl alcohol and polyvinyl pyrrolidone.

153. A prosthetic nucleus apparatus, as in claim 151, wherein said hydrogel comprises polyethylene glycol.

154. A prosthetic nucleus apparatus, as in claim 151, wherein said hydrogel comprises polyvinyl pyrrolidone.
